(19) United States
(12) Patent Application Publication
(10) Pub. No.: US 2013/0011841 A1
(43) Pub. Date: Jan. 10, 2013

(54) Genetic Association of Polymorphisms in Perilipin (PLIN) Gene With Resistance to Weight Loss

(76) Inventors: Nazneen Aziz, Lexington, MA (US); Prakash Prabhakar, Braintree, MA (US); Venkateswarlu Kondragunta, Woburn, MA (US); Jose Ordovas, Framingham, MA (US); Susan B. Roberts, Weston, MA (US); Sai Krupa Das, Boston, MA (US)

(21) Appl. No.: 13/615,204

(22) Filed: Sep. 13, 2012

Related U.S. Application Data
(63) Continuation of application No. 12/833,467, filed on Jul. 9, 2010.
(60) Provisional application No. 61/224,131, filed on Jul. 9, 2009.

(51) Int. Cl. C12Q 1/68; G01N 21/64

(52) U.S. Cl. 435/6.11

(57) ABSTRACT

Diagnostics and therapeutics for resistance to weight-loss, which are based upon the identification of a subject's PLIN polymorphisms, haplotype and genotype pattern, are described in this invention.

### RELATED APPLICATIONS

This application is a continuation of U.S. application Ser. No. 12/833,467, filed on Jul. 9, 2010, which claims the benefit of the filing date of U.S. Provisional Patent Application No. 61/224,131, filed on Jul. 9, 2009, each of which is incorporated herein by reference in its entirety.

### FIELD OF THE INVENTION

This application relates to methods of determining a subject's metabolic genotype and methods for selecting an appropriate therapeutic/dietary regimen or lifestyle recommendation based on subject's genetic profile and susceptibility to adverse weight management issues.

### BACKGROUND

According to a report published in 1998 by the World Health Organization (WHO), obesity has reached epidemic proportions worldwide: about 1.7 billion people worldwide are overweight and 300 million of them are obese. In the U.S., approximately 127 million adults are overweight and 69 million are obese. Obese subjects are at increased risk of developing one or more serious medical conditions including diabetes, heart disease, high blood pressure and high blood cholesterol. The prevalence of obesity as more than doubled in the past 25 years and now reaches 31% among U.S. adults aged 20 years and older. Higher rates of obesity are seen among African-Americans and Hispanic Americans, especially among women (30% to 50%).

The increase in the prevalence of obesity observed worldwide in the past decades has occurred in a changing environment characterize by a progressive reduction of physical activity level and the abundance of highly palatable foods. The WHO Report identified these changes as the two principal modifiable characteristics of modern lifestyle promoting the development of obesity. However, despite the fact that people are exposed to the same environment, not everyone is becoming obese, suggesting a role for a subject's genetic profile in the development of weight management issues. That is, genetics determines a subject's susceptibility to become obese when exposed to a unfavorable environment as well as the way he/she can respond to diet and exercise.

Accordingly, there is a need for a means for establishing a personalized weight loss program that considers a person's genetic susceptibility to obesity in order to improve weight loss and weight maintenance outcomes relative to a similar program not taking into account of the genetic information. There is a need for means for linking a subject's metabolic genotype to response to diet and/or exercise.

Perilipin is one of a family of proteins in adipocytes that function to regulate cellular triglyceride storage and mobilization. Perilipin knockout mice are lean and resistant to diet-induced obesity. In this regard, adipose tissue plays a central role in regulating energy storage and mobilization, and it has been the focus of efforts to identify candidate genes for obesity and weight management. Perilipins are phosphorylated proteins in adipocytes that are localized at the surface of the lipid droplet. Experimental studies have shown that these proteins are essential in the regulation of triglycerides deposition and mobilization. After activation of protein kinase A, perilipin is phosphorylated, resulting in translocation of the protein away from the lipid droplet and allowing hormone-sensitive lipase to hydrolyze the adipocyte triglycerides to release non-esterified fatty acids. Perilipin functions to increase cellular triglycerides storage by decreasing the rate of triglycerides hydrolysis and serves an additional role in controlling the release of triglycerides at times of need.

The description herein of disadvantages and problems associated with known methods is in no way intended to limit the scope of the embodiments described in this document to their exclusion.

The following published patent applications describe a variety of methods for determining a subject's metabolic genotype and for personalize diet design based on a subject's predicted likely response to weight loss and weight management based on genetic polymorphisms in the perilipin (PLIN) gene: WO 2007/027229, US 2006/0252050 A1 and US 2007/0248959 A1.

Genotype Screening

Traditional methods for the screening of heritable diseases have depended on either the identification of abnormal gene products (e.g., sickle cell anemia) or an abnormal phenotype (e.g., mental retardation). These methods are of limited utility for heritable diseases with late onset and no easily identifiable phenotypes such as, for example, vascular disease. With the development of simple and inexpensive genetic screening methodology, it is now possible to identify polymorphisms that indicate a propensity to develop disease, even when the disease is of polygenic origin. The number of diseases that can be screened by molecular biological methods continues to grow with increased understanding of the genetic basis of multifactorial disorders.

Genetic screening (also called genotyping or molecular screening), can be broadly defined as testing to determine if a patient has mutations (alleles or polymorphisms) that either cause a disease state or are “linked” to the mutation causing a disease state. Linkage refers to the phenomenon that DNA sequences which are close together in the genome have a tendency to be inherited together. Two sequences may be linked because of some selective advantage of co-inheritance. More typically, however, two polymorphic sequences are co-inherited because of the relative infrequency with which meiotic recombination events occur within the region between the two polymorphisms. The co-inherited polymorphic alleles are said to be in linkage disequilibrium with one another because, in a given human population, they tend to either both occur together or else not occur at all in any particular member of the population. Indeed, where multiple polymorphisms in a given chromosomal region are found to be in linkage disequilibrium with one another, they define a quasi-stable genetic “haplotype.” In contrast, recombination events occurring between two polymorphic loci cause them to become separated onto distinct homologous chromosomes. If meiotic recombination between two physically linked polymorphisms occurs frequently enough, the two polymorphisms will appear to segregate independently and are said to be in linkage equilibrium.

While the frequency of meiotic recombination between two markers is generally proportional to the physical distance between them on the chromosome, the occurrence of “hot spots” as well as regions of repressed chromosomal recombination can result in discrepancies between the physical and recombinational distance between two markers. Thus, in certain chromosomal regions, multiple polymorphic loci spanning a broad chromosomal domain may be in linkage disequilibrium with one another, and thereby define a broad-spanning genetic haplotype. Furthermore, where a disease-causing mutation is found within or in linkage with this haplotype, one or more polymorphic alleles of the haplotype can be used as a diagnostic or prognostic indicator of the likelihood of developing the disease. This association between otherwise benign polymorphisms and a disease-causing polymorphism occurs if the disease mutation arose in the recent past, so that sufficient time has not elapsed for equilibrium to be achieved through recombination events. Therefore identification of a human haplotype which spans or is linked to a disease-causing mutational change, serves as a predictive measure of a subject's likelihood of having inherited that disease-causing mutation. Importantly, such prognostic or diagnostic procedures can be utilized without necessitating the identification and isolation of the actual disease-causing lesion. This is significant because the precise determination of the molecular defect involved in a disease process can be difficult and laborious, especially in the case of multifactorial diseases such as inflammatory disorders.

Indeed, the statistical correlation between obesity and PLIN polymorphism does not necessarily indicate that the polymorphism directly causes the disorder. Rather the correlated polymorphism may be a benign allelic variant which is linked to (i.e. in linkage disequilibrium with) a disorder-causing mutation which has occurred in the recent human evolutionary past, so that sufficient time has not elapsed for equilibrium to be achieved through recombination events in the intervening chromosomal segment. Thus, for the purposes of diagnostic and prognostic assays for a particular disease, detection of a polymorphic allele associated with that disease can be utilized without consideration of whether the polymorphism is directly involved in the etiology of the disease. Furthermore, where a given benign polymorphic locus is in linkage disequilibrium with an apparent disease-causing polymorphic locus, still other polymorphic loci which are in linkage disequilibrium with the benign polymorphic locus are also likely to be in linkage disequilibrium with the disease-causing polymorphic locus. Thus these other polymorphic loci will also be prognostic or diagnostic of the likelihood of having inherited the disease-causing polymorphic locus. Indeed, a broad-spanning human haplotype (describing the typical pattern of co-inheritance of alleles of a set of linked polymorphic markers) can be targeted for diagnostic purposes once an association has been drawn between a particular disease or condition and a corresponding human haplotype. Thus, the determination of a subject's likelihood for developing a particular disease of condition can be made by characterizing one or more disease-associated polymorphic alleles (or even one or more disease-associated haplotypes) without necessarily determining or characterizing the causative genetic variation.

### SUMMARY OF THE INVENTION

The invention provides a genetic predisposition test that allows predicting a subjects likely response to weight loss and specific weight management strategies based on genetic polymorphisms in the perilipin (PLIN) gene. The invention also provides kits to determine whether a subject is resistant to weight gain or weight loss based on analysis of genetic polymorphisms at the perilipin gene. This information can be used to screen subjects, such as obese and overweight subjects and classify them based on their genetic tendency to lose weight more successfully with low calorie diets that are reduced in fat (Low Fat); low calorie diets that are reduced in carbohydrates (Low Carb); liquid diets that are very low in calories as well as very low in fat and carbohydrates (Very Low Cal). Appropriate measure can then be implemented in life-style, diet, medicinal and possible surgical interventions. Such a genetic approach will help professionals in the field of weight-management to improve targeting patients with appropriate advice regarding their weight management.

The primary goal of this test is to classify overweight and obese individuals based on PLIN gene polymorphisms (Listed in Table 8), both individually and in combination, to inform weight loss decisions and improve weight management.

In one aspect, the presence, absence or predisposition to more successful weight loss in a subject is determined by detecting in the subject genotype that is associated with more predictable weight loss under specific dietary conditions. The presence of the genotype indicates that the subject has or is predisposed to resistance to weight loss in general, thereby requiring more extreme caloric reduction, or is predisposed to lose weight more predictably under certain dietary conditions—i.e. Low Fat or Low Carb diets. In contrast, absence of the genotype indicates that the subject does not have or is not predisposed to resistance to weight loss. A symptom of resistance to weight loss is alleviated by detecting the presence of a weight loss associated genotype and guiding medical management of obese patients with recommendations for specific diet, exercise, therapeutics, or other medical interventions that are currently used to treat the major complications of obesity, particularly metabolic syndrome and fatty liver/non-alcoholic steatohepatitis (NASH).

The invention is based on the finding that obese carriers of the PLIN 4 allele A and/or PLIN Z allele A were less likely to lose weight on a diet with moderate calorie restriction (Group A) compared to liquid diet that was very low in calories and very low in fats (1,000 to 1,200 kcal; Group BC). The invention also is based on the finding that obese carriers of the PLIN 4 allele A were more successful in weight loss on diets that were very low calorie and very low fat and carriers of the PLIN 4 genotype GG were successful in weight loss even on diets that were moderate calorie restriction.

Accordingly, the invention provides a method of predicting a subject's response to a weight management program the method comprising analyzing the subject's genotype at the perilipin gene, wherein the presence of either one or two alleles of PLIN 4 SNP rs894160 (A); one or two alleles of PLIN Z SNP rs8179043 (A); and/or one or two alleles of PLIN 1 SNP rs2289487 (G) is indicative of the subject being likely resistant to weight change unless the diet is very low calorie and/or very low fat, whereas subjects with the other PLIN genotypes will respond favorably to other dietary configurations.

In one embodiment, the invention provides a method of determining whether an overweight or obese subject is a suitable candidate, i.e., susceptible for weight-loss program, or for weight-management program comprising a dietary component alone or as its main component, for example, low energy diet also called low calorie diet. The method comprises genotyping the PLIN gene, preferably at PLIN 4, PLIN Z and/or PLIN 1 loci, of the overweight or obese subject, wherein the absence of one or two alleles of PLIN 4 SNP rs894160 (A); one or two alleles of PLIN Z SNP rs8179043 (A); and/or one or two alleles of PLIN 1 SNP rs2289487 (G) is indicative of the subject being a good candidate for weight-management by low-energy diet.

In some embodiments of the invention, the invention provides a method for determining whether an overweight or obese subject is not a suitable candidate, i.e., susceptible for weight loss, for weight-management program comprising a dietary component, such as low-energy or low calorie diet. The method comprises genotyping the PLIN gene, preferably at PLIN 4, PLIN Z and/or PLIN 1 loci, of the overweight or obese subject, wherein the presence of PLIN 4 SNP rs894160 (A); one or two PLIN Z SNP rs8179043 (A); and/or one or two PLIN 1 SNP rs2289487 (G) loci is indicative of the subject not being a good candidate for weight-management by a low-energy diet alone.

In some embodiments of the invention, the invention provides a kit for determining whether a subject is an appropriate candidate for a weight-management program, preferably to a program that comprises a dietary intervention component, for example, low-energy diet, wherein the kit comprises genotyping means for PLIN gene, preferably at least PLIN 4, PLIN Z and PLIN 1 loci, or any other PLIN locus in linkage disequilibrium with PLIN 4, PLIN Z or PLIN 1 locus, and an instruction manual explaining that detection of at least one allele A at PLIN 4 locus; allele A at PLIN Z locus; or allele G at PLIN 1 locus is indicative of the subject as being less likely to be successful in weight-management by dietary, particularly low-calorie, interventions.

In some embodiments of the invention, detection of other than “A” allele for PLIN 4 and PLIN Z and “A” for PLIN 1, such as detection of a subject homozygous for “G” allele for PLIN 4; “G” allele of PLIN Z; and/or “G” allele for PLIN 1, is indicative of that subject being susceptible, i.e., a good candidate for weight-management using dietary, for example, low-calorie intervention either alone or as one major component of the weight-management program.

In some embodiments of the invention, the invention provides a method for selecting among therapeutic/surgical/dietary or lifestyle options in overweight or obese subjects with genotype in PLIN 1 (rs2289487; G/*), PLIN 4 (rs894160; A/A), and PLIN Z (rs8179043; A/A) SNPs on the perilipin gene, wherein the presence of any one, any two or all three genotypes will indicate that subject will be less successful in weight loss when prescribed a calorie restricted diet.

In some embodiment of the invention, the invention provides a method of selecting patients for clinical trials for weight management therapies based on identification of a subject's genotype at PLIN 1 (rs2289487), PLIN 4 (rs894160) and PLIN Z (rs8179043) loci on the perilipin gene.

In some embodiments of the invention, the invention provides a method for selecting the responders of bariatric surgery based on identification of a subject's genotype at PLIN 1 (rs2289487), PLIN 4 (rs894160) and PLIN Z (rs8179043) loci on the perilipin gene. Subjects who lose weight on the calorie restricted diets prior to bariatric surgery are more likely to maintain more weight loss after the surgery (Still et. al, Arch Surg. 2007; 142(10):994-998).

In some embodiments of the invention, the invention provides a method for determining subject/subjects who will lose more body fat based on identification of a subject's genotype at PLIN 1 (rs2289487), PLIN 4 (rs894160) and PLIN Z (rs8179043) loci on the perilipin gene. Perilipin affects fat metabolism and subjects who are resistant to weight loss will have higher body fat content.

In some embodiments of the invention, the invention provides a method for selecting therapeutic/surgical/dietary or lifestyle in overweight or obese subjects carrying haplotype patterns AAG or GGA, consisting of PLIN 4 (rs894160), PLIN Z (rs8179043) and PLIN 1 (rs2289487) and haplotype patterns GGAG and AAGG consisting of PLIN 4 (rs894160), PLIN Z (rs8179043), PLIN 1 (rs2289487), PLIN X (rs4578621) in the perilipin gene, wherein the presence of any one, any two or all four haplotypes will indicate that subject will be resistant to weight loss when prescribed a calorie restricted diet.

In some embodiments of the invention, the invention provides a method of selecting patients for clinical trials for weight management therapies based on identification of a subject's haplotype patterns at PLIN 4 (rs894160), PLIN Z (rs8179043), PLIN 1 (rs2289487), PLIN X (rs4578621) on the perilipin gene.

In some embodiments of the invention, the invention provides a method for selecting the responders of bariatric surgery based on identification of a subject's haplotype patterns at PLIN 4 (rs894160), PLIN Z (rs8179043), PLIN 1 (rs2289487), PLIN X (rs4578621) on the perilipin gene. Subjects who lose weight on the calorie restricted diets prior to bariatric surgery are more likely to maintain more weight loss after the surgery (Still et. al, Arch Surg. 2007; 142(10):994-998).

In some embodiments of the invention, the invention provides a method for determining subject/subjects who will lose more body fat based on identification of a subject's haplotype patterns at PLIN 4 (rs894160), PLIN Z (rs8179043), PLIN 1 (rs2289487), PLIN X (rs4578621) on the perilipin gene. Perilipin affects fat metabolism and subjects who are resistant to weight loss will have higher body fat content.

In some embodiments of the invention, the invention provides a method for selecting among therapeutic/surgical/dietary or lifestyle options in overweight or obese subjects with genotype in PLIN 1 (rs2289487; G/*), PLIN 4 (rs894160; A/A) and PLIN Z (rs8179043; A/A) SNPs on the perilipin gene, wherein the presence of any one, any two or all three genotypes will indicate that subject will be resistant to weight loss when prescribed a calorie restricted diet.

The present invention further provides two novel polymorphisms to assist in advising obese or overweight individuals with choosing a more effective weight loss program. A carrier of PLIN5 “G” or “C” allele (rs2304795), depending on which DNA strand is analyzed, is more likely to be successful in weight loss with a low complex carbohydrate diet, whereas an individual carrying a PLIN6 “T” or “A” allele (rs1052700), depending on which DNA strand is analyzed, will have better success with a high complex carbohydrate diet.

Accordingly, the invention provide a method for determining an optimal weight management program for an obese or overweight individual carrying at least one “C” allele at perilipin locus rs2304795 (PLIN5; C>T), the method comprising the step of prescribing or advising the individual to follow a low glycemic diet to an individual carrying at least one “C” allele at perilipin locus rs2304795. If the analysis is performed using the opposite DNA strand, the allele associated with this embodiment naturally comprises a “G” allele.

In one embodiment of this aspect and all other aspects disclosed herein, the method further comprises a step of first determining the individual's genotype at the perilipin locus rs2304795.

Another aspect described herein is a method for assisting in reducing weight in an overweight or obese individual carrying at least one “C” allele at perilipin locus rs2304795, the method comprising the step of prescribing or advising the individual to follow a low carbohydrate diet to the individual, wherein the low carbohydrate diet prescribed to the individual assists in a reduction in weight of the individual. If the analysis is performed using the opposite DNA strand, the allele associated with this embodiment naturally comprises a “G” allele.

In one embodiment of this aspect and all other aspects disclosed herein, the method further comprises a step of first determining the individual's genotype at said perilipin locus rs2304795.

Another aspect described herein is a method to assist in decreasing body fat composition in an obese or overweight individual carrying at least one “C” allele at perilipin locus rs2304795, the method comprising the step of prescribing or advising the individual to follow a low carbohydrate diet to an individual carrying at least one “C” allele at perilipin locus rs2304795, wherein the low carbohydrate diet prescribed to the individual assists in decreasing body fat composition of the individual. If the analysis is performed using the opposite DNA strand, the allele associated with this embodiment naturally comprises a “G” allele.

In one embodiment of this aspect and all other aspects disclosed herein, the method further comprises a step of first determining the individual's genotype at the perilipin locus rs2304795.

Another aspect disclosed herein is a method to assist in improving metabolic rate in an overweight or obese individual carrying at least one “C” allele at perilipin locus rs2304795, the method comprising the step of prescribing or advising the individual to follow a low carbohydrate diet to an individual carrying at least one “C” allele at perilipin locus rs2304795, wherein the low carbohydrate diet prescribed to the individual assists in improving metabolic rate of the individual. If the analysis is performed using the opposite DNA strand, the allele associated with this embodiment naturally comprises a “G” allele.

In one embodiment of this aspect and all other aspects disclosed herein, the method further comprises a step of first determining the individual's genotype at the perilipin locus rs2304795.

Another aspect disclosed herein is a method for determining an optimal weight management program for an obese or overweight individual carrying at least one “T” allele at perilipin locus rs1052700, the method comprising the step of prescribing or advising the individual to follow a high complex carbohydrate diet to an individual carrying at least one “T” allele at perilipin locus rs1052700. If the analysis is performed using the opposite DNA strand, the allele associated with this embodiment naturally comprises an “A” allele.

In one embodiment of this aspect and all other aspects disclosed herein, the method further comprises a step of first determining the individual's genotype at the perilipin locus rs1052700.

Another aspect disclosed herein is a method for assisting in reducing weight in an overweight or obese individual carrying at least one “T” allele at perilipin locus rs1052700, the method comprising the step of prescribing or advising the individual to follow a high complex carbohydrate diet to the individual, wherein the high complex carbohydrate diet prescribed to the individual assists in a reduction in weight of the individual. If the analysis is performed using the opposite DNA strand, the allele associated with this embodiment naturally comprises an “A” allele.

In one embodiment of this aspect and all other aspects disclosed herein, the method further comprises a step of first determining the individual's genotype at said perilipin locus rs1052700.

Another aspect disclosed herein is a method to assist in decreasing body fat composition in an obese or overweight individual carrying at least one “T” allele at perilipin locus rs1052700, the method comprising the step of prescribing or advising the individual to follow a high complex carbohydrate diet to an individual carrying at least one “T” allele at perilipin locus rs1052700, wherein the high complex carbohydrate diet prescribed to the individual assists in decreasing body fat composition of the individual. If the analysis is performed using the opposite DNA strand, the allele associated with this embodiment naturally comprises an “A” allele.

In one embodiment of this aspect and all other aspects disclosed herein, the method further comprises a step of first determining the individual's genotype at the perilipin locus rs1052700.

Another aspect disclosed herein is a method for determining an optimal weight management program for an obese or overweight individual carrying at least one “G” allele at perilipin locus rs894160, the method comprising the step of prescribing or advising the individual to follow a low glycemic diet to the individual carrying at least one “G” allele at perilipin locus rs894160. If the analysis is performed using the opposite DNA strand, the allele associated with this embodiment naturally comprises a “G” allele.

In one embodiment of this aspect and all other aspects disclosed herein, the method further comprises a step of first determining the individual's genotype at the perilipin locus rs894160.

Another aspect described herein is a method for assisting in reducing weight in an overweight or obese individual carrying at least one “G” allele at perilipin locus rs2289487, the method comprising the step of prescribing or advising the individual to follow a low carbohydrate diet to an individual carrying at least one “G” allele at perilipin locus rs2289487, wherein the low carbohydrate diet prescribed to the individual assists in a reduction in weight of the individual. If the analysis is performed using the opposite DNA strand, the allele associated with this embodiment naturally comprises a “C” allele.

In one embodiment of this aspect and all other aspects disclosed herein, the method further comprises a step of first determining the individual's genotype at the perilipin locus rs2289487.

Also described herein is a method to assist in decreasing body fat composition in an obese or overweight individual carrying at least one “G” allele at perilipin locus rs2289487, the method comprising the step of prescribing or advising the individual to follow a low carbohydrate diet to the individual carrying at least one “G” allele at perilipin locus rs2289487, wherein the low carbohydrate diet prescribed to the individual assists in decreasing body fat composition of the individual. If the analysis is performed using the opposite DNA strand, the allele associated with this embodiment naturally comprises a “C” allele.

In one embodiment of this aspect and all other aspects disclosed herein, the method further comprises a step of first determining the individual's genotype at the perilipin locus rs2289487.

Also described herein is a method to assist in improving metabolic rate in an overweight or obese individual carrying at least one “G” allele at perilipin locus rs2289487, the method comprising the step of prescribing or advising the individual to follow a low carbohydrate diet to an individual carrying at least one “G” allele at perilipin locus rs2289487, wherein the low carbohydrate diet prescribed to the individual assists in improving metabolic rate of the individual. If the analysis is performed using the opposite DNA strand, the allele associated with this embodiment naturally comprises a “G” allele.

In one embodiment of this aspect and all other aspects described herein, the method further comprises a step of first determining the individual's genotype at the perilipin locus rs2289487.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is an illustration showing overall Study Design in the current invention.

FIG. 2A is a drawing of the position of all the tested SNPs on PLIN gene.

FIG. 2B is a drawing of the LD analysis of SNPs in PLIN gene shown in FIG. 2A. PLIN 1, 4 and Z, SNPs showed strong LD.

FIG. 3 is a graph showing % weight change during caloric restriction in groups randomly assigned to consume a diet with either a high glycemic load or a low glycemic load.

FIG. 4A is a line graph showing amount of weight change at one year based on PLIN genotype and number of minor alleles.

FIG. 4B is a line graph showing % change in body fat at one year based on PLIN genotype and number of minor alleles.

FIG. 4C is a line graph showing negative change in metabolic rate at one year based on PLIN genotype and number of minor alleles.

FIG. 5A shows a bar graph depicting mean weight loss for individuals having a PLIN1 polymorphism at one year of groups randomly assigned to a high glycemic or low glycemic diet.

FIG. 5B shows a bar graph depicting mean body fat mass loss for individuals having a PLIN1 polymorphism at one year of groups randomly assigned to a high glycemic or low glycemic diet.

FIG. 6A shows a bar graph depicting mean weight loss for individuals having a PLIN4 polymorphism at one year of groups randomly assigned to a high glycemic or low glycemic diet.

FIG. 6B shows a bar graph depicting mean body fat mass loss for individuals having a PLIN4 polymorphism at one year of groups randomly assigned to a high glycemic or low glycemic diet.

FIG. 7 is a bar graph showing waist size for individuals with a PLIN4 polymorphism and randomly assigned to a low complex carbohydrate or a high complex carbohydrate diet.

FIG. 8 is a line graph showing the effects of a PLIN4 polymorphism on predicted waist size based on a diet with varying amounts of complex carbohydrate.

### DETAILED DESCRIPTION OF THE INVENTION

The invention bases upon the discovery of genotypes associated with resistance to weight-loss. Accordingly, the invention provides a genetic predisposition test that identifies a subject with elevated risk for lack of response to dietary regimen directed to weight-loss.

According to some embodiment of the invention, the presence, absence or predisposition to resistance to weight loss in a subject is determined by detecting in the subject a resistance to weight-loss-associated genotype. The presence of the genotype indicates that the subject has or is predisposed to resistance to weight loss.

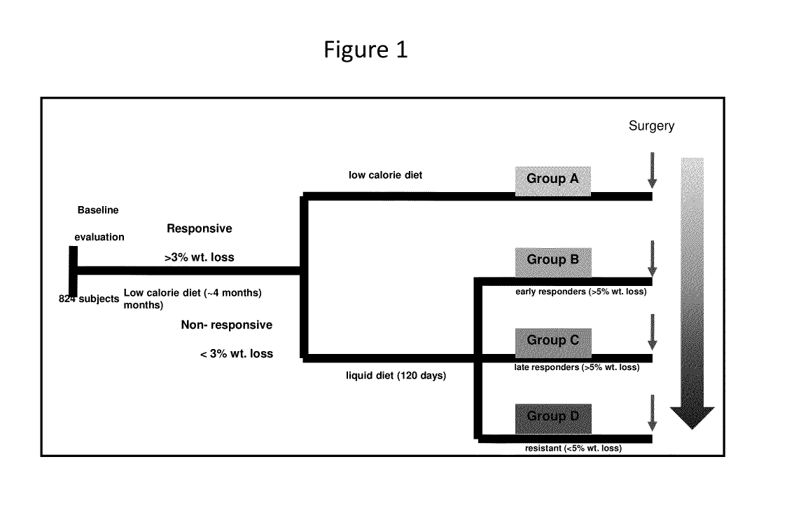

According to some embodiments of the invention, a subject who lost about 3% or >3% of weight (total body weight measure) after about 4 months being enrolled in a low calorie diet (for example, about 1200-1500 kcal for women and 1500-1800 for men) were considered to have lost weight in response to low calorie diet in stage 1 and were classified as Group A (FIG. 1). In stage 2 (after the first 4 months, another about 4 months) all subjects who lost <3% weight in stage 1 were recommended a liquid diet of 1000 kcal (women) or 1200 kcal (men). Once on liquid diet, subjects who lost 5% or >5% of total body weight in an early stage were classified as Group B (early responders), and those who lost the same amount of weight, but at a later stage were categorized in Group C (late responders). Subjects, who did not respond to either stages (I or II), were classified as Group D (non-responders). (FIG. 1.)

According to some embodiments, the Group B early responders responded to liquid diet between 20-30 days, or 31-40 days, or 41-50 days, or 51-60 days, or 61-70 days, or 71-80 days, or 81-90 days, or 91-100 days, or 101-110 days, or 111-120 days. In some preferred embodiments, the Group B early responders responded between 20-120 days, or 20-60 days, or 30-60 days, or 30-120 days, or 60-120 days.

According to some embodiments, the Group C late responders responded to liquid diet between 120-130 days, or 131-140 days, or 141-150 days, or 151-160 days, or 161-170 days, or 171-180 days, or 181-190 days, or 191-200 days, or 201-210 days, or 211-220 days, or 221-230 days, or 231-240 days, or 241-250 days, or 251-260 days, or 261-270 days, or 271-280 days, or 281-290 days, or 291-300 days, or 301-310 days, or 311-320 days, or 321-330 days, or 331-340 days, or 341-350 days, or 351-360 days, or 361-370 days. In some preferred embodiments, the Group C late responders respond between 121-190 days, or 121-360 days, or 121-370 days, or 121-180 days, or 121-220 days, or 121-160 days, or 160-200 days, or 160-180 days, or 160-220 days, or 180-220 days, or 180-370 days.

According to some embodiments, methods are provided for screening subjects of the general population, such as teenagers or normal weight adults, who may be overly conscious of their weight, even if it falls into the so called “normal” range, which is BMI 18.5-24.9. According to this invention, an underweight subject has a BMI<18.5; an overweight subject in the range 25-29.9, an obese subject has a BMI of 30-39.9, and BMI of >40.0 is considered extremely obese. Identification of metabolic genotype in these subjects could provide health professionals with tools to better guide healthy weight management by prescribing specific dietary modifications based on the perilipin genotype.

According to some embodiments, methods and kits are provided for screening subjects for clinical trials for weight management, wherein an underweight subject has a BMI<18.5; an overweight subject in the range 25-29.9, an obese subject has a BMI of 30-39.9, and BMI of >40.0 is considered extremely obese. Identification of metabolic genotype in these subjects could provide health professionals with tools to better guide healthy weight management by prescribing specific dietary modifications based on the perilipin genotype.

A genetic analysis is conducted herein on the association of resistance to weight loss correlated to the occurrence of gene polymorphisms, including, inter alia, certain alleles of the perilipin gene. Investigation of genetic influences on resistance to weight-loss by investigating gene polymorphisms in an obese subject is useful in assessing the clinical utility of employing genetic tests for identifying subjects at high risk in order to target preventative therapies.

Perilipin (PLIN) is a hormonally-regulated phosphoprotein that encircles the lipid storage droplet in adipocytes (Greenberg, A. S.; Egan, J. J.; Wek, S. A.; Takeda, T.; Londos, C.; Kimmel, A. K. (Abstract) Clin. Res. 39: 287A only, 1991). It is the major cellular Protein kinase A (PKA) substrate in adipocytes that coats intracellular lipid droplets and modulates adipocyte lipolysis activity. Nishiu et al. cloned a cDNA encoding human perilipin from an adipose tissue cDNA library (Genomics 48: 254-257, 1998; GenBank Nucleic Acid ID No. gi:3041770). The human gene encodes a 522-amino acid polypeptide that is 79% identical to the rat homolog isolated by Greenberg et al. (Proc. Nat. Acad. Sci. 90: 12035-12039, 1993).

One objective of this study is to determine whether specific variations in perilipin gene may be used to predict the risk of an overweight subject. The invention is based on the finding that subjects carrying certain PLIN alleles were found to be resistant to weight loss compared to subjects carrying other PLIN alleles. The current invention examined the association of polymorphisms at the perilipin (PLIN) gene (PLIN 1: rs2289487; PLIN 4: rs894161; PLIN Z: rs8179043; PLIN X: rs4578621; PLIN 6: rs1052700; PLIN Y: rs894161; and PLIN5: rs2304795) with obesity and weight reduction in response to low-energy diet in morbidly obese patients (initial body mass index between 48-51 kg/m2). The PLIN 4 genotype AA (rs2289487); PLIN Z genotype AA (rs8179043) revealed statistically significant association with resistance to weight loss on diets with moderate calorie restriction (Group A) compared to a very low calorie liquid diet with very low levels of fats (1,000 to 1,200 kcal; Group BC). The PLIN 1 genotype G/* (rs2289487) showed statistically significant association with resistance to weight loss when responders versus non-responders were compared in Group A versus D (FIG. 1).

Consequently, the identification of the PLIN 4 genotype AA (rs2289487); PLIN Z genotype AA (rs8179043); and PLIN 1 genotype G/* (rs2289487) alleles would help weight management professionals to design alternative weight management programs for these subjects. Alternatives to low-energy diets include very low calorie liquid diets that are high in protein and very low in fats, drug treatment, particularly drugs increasing energy consumption rather that limiting energy absorption, surgery, dietary supplements and liposuction. Subjects carrying PLIN 4 genotype AA (rs2289487); PLIN Z genotype AA (rs8179043); and PLIN 1 genotype G/* (rs2289487) alleles would likely benefit from a combination of one or more of the methods listed above either with or without a low-energy diet.

In some embodiments of the invention, the invention provides for a method of determining resistance to weight loss, in response to low calorie diet, in a subject comprising the steps of: (a) providing a biological sample comprising genomic DNA from said subject; (b) detecting, in said DNA, one or more of the following risk alleles, selected from: (i) rs894160 (A) of PLIN 4; (ii) rs8179043 (A) of PLIN Z; and (iii) rs2289487 (A) of PLIN 1; wherein the presence of said risk allele indicates said subject is non-responsive to diet with low caloric restriction.

In some embodiments of the invention, the invention provides creating a haplotype or allelic pattern based on the PLIN genotypes comprising detecting in said subject's DNA the following haplotype patterns: (i) A (rs894160 of) PLIN 4, A (rs8179043 of PLIN Z), G (rs2289487 of PLIN 1); (ii) G (rs894160 of) PLIN 4, G (rs8179043 of PLIN Z), A (rs2289487 of PLIN 1); (iii) G (rs894160 of) PLIN 4, G (rs8179043 of PLIN Z), A (rs2289487 of PLIN 1), G (rs4578621 of PLIN X); (iv) A (rs894160 of) PLIN 4, A (rs8179043 of PLIN Z), G (rs2289487 of PLIN 1), G (rs4578621 of PLIN X); wherein the presence of any one, any two, any three or all four haplotype patterns indicates said subject is non-responsive to diet with low caloric restriction.

The method of the present invention can also be used in screening subjects of the general population, such as teenagers, who may be overly conscious of their weight, even if it falls into the so called “normal” range, one definition of which is BMI 18.5-24.9. Identification of PLIN 4 genotype AA (rs2289487); PLIN Z genotype AA (rs8179043); and PLIN 1 genotype AA (rs2289487) alleles in these subjects could provide health professionals with tools to better guide healthy weight management by prescribing specific dietary modifications based on the perilipin genotype.

Perilipin gene is present on chromosome 15 in the human genome. The perilipin or PLIN locus as used herein refers to loci including, but not limited to PLIN 1 (rs2289487) at nucleotide position 88018100 on chromosome 15 (Genomic build 36.3), PLIN 4 (rs894160) at nucleotide position 88012827 on chromosome 15 (Genomic build 36.3), PLIN5 (rs2304795) at nucleotide position 88011267 on chromosome 15 (Genomic build 36.3), PLIN Z (rs8179043) at nucleotide position 88013148 on chromosome 15 (Genomic build 36.3), PLIN X (rs4578621) at nucleotide position 88022958 on chromosome 15 (Genomic build 36.3), and PLIN 6 (rs1052700) at nucleotide position 88009314 on chromosome 15 (Genomic build 36.3).

One particularly useful locus in the method according to the present invention is the PLIN 4 locus or any other locus in very tight linkage disequilibrium with the PLIN 4 such as PLIN Z and PLIN 1 loci. As used herein, a “very tight linkage disequilibrium” means a polymorphic marker that co-segregates 100% with the PLIN 1 (rs2289487), PLIN 4 (rs894160) and PLIN Z (rs8179043) loci on the perilipin gene. Therefore, any tightly linked polymorphic marker discovered by in-silico searches or by re-sequencing of carriers of the PLIN 4 locus could be also used as diagnostic tools.

According to some embodiments, methods are provided for determining if a subject is resistant to achieve weight loss, comprising detecting in said subject's DNA the following risk alleles: (i) rs894160 (A) of PLIN 4; (ii) rs8179043 (A) of PLIN Z; and (iii) rs2289487 (A) of PLIN 1; wherein the presence of said risk allele indicates said subject is non-responsive to diet with low caloric restriction.

According to some embodiments, methods are provided for determining if a subject is resistant to achieve weight-loss comprising detecting in said subject's DNA the following risk alleles: (i) homozygous at rs894160 (A/A) of PLIN 4; (ii) homozygous at rs8179043 (A/A) of PLIN Z; and (iii) homozygous at rs2289487 (G/*) of PLIN 1; wherein the presence of said risk allele indicates said subject is non-responsive to diet with low caloric restriction.

According to some embodiments, methods are provided for determining if a subject is resistant to achieve weight-loss comprising detecting in said subject's DNA the following risk alleles: (i) heterozygous at rs894160 (A/) of PLIN 4; (ii) heterozygous at rs8179043 (A/) of PLIN Z; and (iii) rs2289487 (G/* of PLIN 1; wherein the presence of said risk allele indicates said subject is non-responsive to diet with low caloric restriction.

According to some embodiments, methods are provided for determining if a subject is responsive to achieve weight loss comprising detecting in said subject's DNA the following responsive alleles: (i) rs894160 (G) of PLIN 4; (ii) rs8179043 (G) of PLIN Z; and (iii) rs2289487 (A) of PLIN 1; wherein the presence of said responsive allele indicates said subject is responsive to diet with low caloric restriction.

According to some embodiments, methods are provided for determining if a subject is responsive to achieve weight-loss further comprises: determining whether said subject has a genotype comprising any one of the following alleles: (i) homozygous at rs894160 (G/G) of PLIN 4; (ii) homozygous at rs8179043 (G/G) of PLIN Z; and (iii) homozygous at rs2289487 (A) of PLIN 1; wherein the presence of said responsive allele indicates said subject is responsive to diet with low caloric restriction.

According to some embodiments, methods are provided for determining if said subject is responsive to achieve weight-loss further comprises: determining whether said subject has a genotype comprising said risk alleles selected from the group consisting of: (i) heterozygous at rs894160 (G/G) of PLIN 4; (ii) heterozygous at rs8179043 (G/G) of PLIN Z; and (iii) heterozygous at rs2289487 (A/A) of PLIN 1; wherein the presence of said responsive allele indicates said subject is responsive to diet with low caloric restriction.

In some embodiments of the invention, the invention provides creating a haplotype or allelic pattern based on the PLIN genotypes comprising detecting in said subject's DNA the following haplotypes: (i) A (rs894160 of) PLIN 4, A (rs8179043 of PLIN Z), G (rs2289487 of PLIN 1); (ii) G (rs894160 of) PLIN 4, G (rs8179043 of PLIN Z), A (rs2289487 of PLIN 1); (iii) G (rs894160 of) PLIN 4, G (rs8179043 of PLIN Z), A (rs2289487 of PLIN 1), G (rs4578621 of PLIN X); (iv) A (rs894160 of) PLIN 4, A (rs8179043 of PLIN Z), G (rs2289487 of PLIN 1), G (rs4578621 of PLIN X); wherein the presence of any one, any two, any three or all four haplotype patterns indicates said subject is non-responsive to diet with low caloric restriction.

According to some embodiments, methods are provided for selecting an appropriate therapeutic/dietary regimen or lifestyle recommendations for a subject comprising: identifying in a subject's DNA the genotype at any one of loci: the PLIN 1 (rs2289487), PLIN 4 (rs894160; A) and PLIN Z (rs8179043; A), at the perilipin gene.

According to some embodiments, methods are provided for selecting an appropriate therapeutic/dietary regimen or lifestyle recommendations for a subject comprising: identifying in a subject's DNA one or more alleles: PLIN 1 (rs2289487; A/A), PLIN 4 (rs894160; G) and PLIN Z (rs8179043; G), on the perilipin gene, wherein the presence of any one, any two, or all three alleles indicates that the subject is responsive to a low calorie diet.

According to some embodiments, methods are provided for selecting an appropriate therapeutic/dietary regimen or lifestyle recommendations for a subject comprises identifying in a subject's DNA one or more risk alleles: PLIN 1 (rs2289487; G/*), PLIN 4 (rs894160; A/A) and PLIN Z (rs8179043; A/A) SNPs on the perilipin gene, wherein the presence of any one, any two or all three risk alleles indicates that subject is less responsive or resistant to weight loss when prescribed a calorie restricted diet.

According to some embodiments, methods are provided for selecting patients for clinical trials for weight management, based on the identification of said patients genotype at the perilipin gene selected from the group consisting of: the PLIN 1 (rs2289487; G), PLIN 4 (rs894160; A) and PLIN Z (rs8179043; A) loci.

According to some embodiments, methods are provided for selecting the responders of bariatric surgery based on the identification of a subject's genotype on the perilipin gene, selected from the group consisting at PLIN 1 (rs2289487), PLIN 4 (rs894160) and PLIN Z (rs8179043) loci.

According to some embodiments, methods are provided for identifying subjects who will likely loose body fat based on the identification of a subject's genotype on the perilipin gene, selected from the group consisting at PLIN 1 (rs2289487), PLIN 4 (rs894160) and PLIN Z (rs8179043) loci.

According to some embodiments, methods are provided for of selecting an appropriate therapeutic/dietary regimen or lifestyle recommendations for a subject comprising detecting in said subject's DNA the following haplotypes: (i) A (rs894160 of) PLIN 4, A (rs8179043 of PLIN Z), G (rs2289487 of PLIN 1); (ii) G (rs894160 of) PLIN 4, G (rs8179043 of PLIN Z), A (rs2289487 of PLIN 1); (iii) G (rs894160 of) PLIN 4, G (rs8179043 of PLIN Z), A (rs2289487 of PLIN 1), G (rs4578621 of PLIN X); (iv) A (rs894160 of) PLIN 4, A (rs8179043 of PLIN Z), G (rs2289487 of PLIN 1), G (rs4578621 of PLIN X); wherein the presence of any one, any two, any three or all four haplotype patterns indicates said subject is non-responsive to diet with low caloric restriction.

According to some embodiments, methods are provided for selecting an appropriate therapeutic/dietary regimen or lifestyle recommendation for a subject comprising genotyping said subject at one or more alleles at: PLIN 4, PLIN Z, PLIN 1, and PLIN 6, wherein the presence of one or more alleles is predictive of said subject's predisposition to weight loss in response to low calorie diet, or liquid diet, or both.

According to some embodiments, methods are provided for selecting an appropriate therapeutic/dietary regimen or lifestyle recommendation for a subject comprises genotyping said subject at the SNP rs894160 of PLIN 4, wherein the presence of homozygous allele A indicates said subject is resistant, and presence of heterozygous allele G indicates said subject is predisposed to respond to weight loss in response to a low calorie diet, or a liquid diet, or both.

According to some embodiments, methods are provided for selecting an appropriate therapeutic/dietary regimen or lifestyle recommendation for a subject comprises genotyping said subject at the SNP rs8179043 of PLIN Z, wherein the presence of homozygous allele A indicates said subject is resistant, and presence of heterozygous allele G indicates said subject is predisposed to respond to weight loss in response to a low calorie diet, or a liquid diet, or both.

According to some embodiments, methods are provided for selecting an appropriate therapeutic/dietary regimen or lifestyle recommendation for a subject comprises genotyping said subject at the SNP rs2289487 of PLIN 1, wherein the presence of homozygous allele A indicates said subject is resistant, and presence of heterozygous allele G indicates said subject is predisposed to respond to weight loss in response to a low calorie diet, or a liquid diet, or both.

According to some embodiments, methods are provided for determining if a subject is resistant to weight loss, comprising genotyping said subject at one or more alleles at: PLIN 4, PLIN Z and PLIN 1, wherein the presence of one or more alleles is predictive of said subject's predisposition to weight loss in response to low calorie diet, or liquid diet, or both.

According to some embodiments, methods are provided for determining if a subject is resistant to weight loss comprises genotyping said subject at the SNP rs894160 of PLIN 4, wherein the presence of homozygous allele A indicates said subject is resistant, and presence of heterozygous allele G indicates said subject is predisposed to respond to weight loss in response to a low calorie diet, or a liquid diet, or both.

According to some embodiments, methods are provided for determining if a subject is resistant to weight loss comprises genotyping said subject at the SNP rs8179043 of PLIN Z, wherein the presence of homozygous allele A indicates said subject is resistant, and presence of heterozygous allele G indicates said subject is predisposed to respond to weight loss in response to a low calorie diet, or a liquid diet, or both.

According to some embodiments, methods are provided for determining if a subject is resistant to weight loss comprises genotyping said subject at the SNP rs2289487 of PLIN 1, wherein the presence of allele G indicates said subject is resistant, and presence of heterozygous allele G indicates said subject is predisposed to respond to weight loss in response to a low calorie diet, or a liquid diet, or both.

According to some embodiments, methods are provided for selecting an appropriate therapeutic/dietary regimen or lifestyle recommendation for a subject, comprising genotyping said subject for composite genotype at one or more alleles at: PLIN 4, PLIN Z, PLIN 1, and PLIN X, wherein the presence of one or more said composite genotypes including said alleles is predictive of said subject's predisposition to weight loss in response to low calorie diet, or liquid diet, or both.

According to some embodiments, methods are provided for selecting an appropriate therapeutic/dietary regimen or lifestyle recommendation for a subject comprises the steps of: a) genotyping said subject at: (i) SNP rs894160 of PLIN 4; (ii) SNP rs8179043 of PLIN Z; and (iii) SNP rs2289487 of PLIN 1; b) determining whether said subject has a composite genotype comprising the allelic pattern or haplotype of: heretozygous allele A at SNP rs894160 of PLIN 4, heterozygous allele A at SNP rs8179043 of PLIN Z, and heterozygous allele G at SNP rs2289487 of PLIN 1; wherein the presence of the haplotype indicates said subject is resistant to weight loss in response to a low calorie or liquid diet.

According to some embodiments, methods are provided for selecting an appropriate therapeutic/dietary regimen or lifestyle recommendation for a subject comprises the steps of: a) genotyping said subject at: (i) SNP rs894160 of PLIN 4; (ii) SNP rs8179043 of PLIN Z; and (iii) SNP rs2289487 of PLIN 1; b) determining whether said subject has a composite genotype comprising the allelic pattern or haplotype of: heretozygous allele G at SNP rs894160 of PLIN 4, heterozygous allele G at SNP rs8179043 of PLIN Z, and heterozygous allele A at SNP rs2289487 of PLIN 1; wherein the presence of the haplotype indicates said subject is resistant to weight loss in response to a low calorie or liquid diet.

According to some embodiments, methods are provided for selecting an appropriate therapeutic/dietary regimen or lifestyle recommendation for a subject comprises the steps of: a) genotyping said subject at: (i) SNP rs894160 of PLIN 4; (ii) SNP rs8179043 of PLIN Z; (iii) SNP rs2289487 of PLIN 1; and (iv) SNP rs4578621 of PLIN X; b) determining whether said subject has a composite genotype comprising the allelic pattern or haplotype of: heterozygous allele G at SNP rs894160 of PLIN 4, heterozygous allele G at SNP rs8179043 of PLIN Z, heterozygous allele A at SNP rs2289487 of PLIN 1 and heterozygous allele G at SNP rs4578621 of PLIN X; wherein the presence of the haplotype indicates said subject is resistant to weight loss in response to a low calorie or liquid diet.

According to some embodiments, methods are provided for selecting an appropriate therapeutic/dietary regimen or lifestyle recommendation for a subject comprises the steps of: a) genotyping said subject at: (i) SNP rs894160 of PLIN 4; (ii) SNP rs8179043 of PLIN Z; (iii) SNP rs2289487 of PLIN 1; and (iv) SNP rs4578621 of PLIN X; b) determining whether said subject has a composite genotype comprising the allelic pattern or haplotype of: heterozygous allele A at SNP rs894160 of PLIN 4, heterozygous allele A at SNP rs8179043 of PLIN Z, heterozygous allele G at SNP rs2289487 of PLIN 1 and heterozygous allele G at SNP rs4578621 of PLIN X; wherein the presence of the haplotype indicates said subject is resistant to weight loss in response to a low calorie or liquid diet.

According to some embodiments, methods are provided for selecting patients for clinical trials comprising genotyping a subject at one or more alleles at: SNP rs894160 of PLIN 4, SNP rs8179043 of PLIN Z, SNP rs2289487 of PLIN 1, SNP rs4578621 of PLIN X, and SNP rs1052700 of PLIN 6; wherein the presence of one or more alleles is predictive of said subject's predisposition to weight loss in response to low calorie diet, or liquid diet, or both.

According to some embodiments, methods are provided for selecting patients for clinical trials comprising genotyping a subject for composite genotype at one or more alleles at: SNP rs894160 of PLIN 4, SNP rs8179043 of PLIN Z, SNP rs2289487 of PLIN 1, SNP rs4578621 of PLIN X, and SNP rs1052700 of PLIN 6; wherein the presence of one or more said composite genotypes including said alleles is predictive of said subject's predisposition to weight loss in response to low calorie diet, or liquid diet, or both.

According to some embodiments, methods are provided for selecting patients for bariatric surgery comprising genotyping a subject at one or more alleles at: SNP rs894160 of PLIN 4, SNP rs8179043 of PLIN Z, SNP rs2289487 of PLIN 1, SNP rs4578621 of PLIN X, and SNP rs1052700 of PLIN 6; wherein the presence of one or more alleles is predictive of said subject's predisposition to weight loss in response to low calorie diet, or liquid diet, or both.

According to some embodiments, methods are provided for selecting patients for bariatric surgery comprising genotyping a subject for composite genotype at one or more alleles at: SNP rs894160 of PLIN 4, SNP rs8179043 of PLIN Z, SNP rs2289487 of PLIN 1, SNP rs4578621 of PLIN X, and SNP rs1052700 of PLIN 6; wherein the presence of one or more said composite genotypes including said alleles is predictive of said subject's predisposition to weight loss in response to low calorie diet, or liquid diet, or both.

The present invention further provides two novel polymorphisms to assist in advising obese or overweight individuals with choosing a more effective weight loss program. A carrier of PLIN5 “G” or “C” allele (rs2304795), depending on which DNA strand is analyzed, is more likely to be successful in weight loss with a low complex carbohydrate diet, whereas an individual carrying a PLIN6 “T” or “A” allele (rs1052700), depending on which DNA strand is analyzed, will have better success with a high complex carbohydrate diet.

Accordingly, the invention provide a method for determining an optimal weight management program for an obese or overweight individual carrying at least one “C” allele at perilipin locus rs2304795 (PLIN5; C>T), the method comprising the step of prescribing or advising the individual to follow a low glycemic diet to an individual carrying at least one “C” allele at perilipin locus rs2304795. If the analysis is performed using the opposite DNA strand, the allele associated with this embodiment naturally comprises a “G” allele.

In one embodiment of this aspect and all other aspects disclosed herein, the method further comprises a step of first determining the individual's genotype at the perilipin locus rs2304795.

Another aspect described herein is a method for assisting in reducing weight in an overweight or obese individual carrying at least one “C” allele at perilipin locus rs2304795, the method comprising the step of prescribing or advising the individual to follow a low carbohydrate diet to the individual, wherein the low carbohydrate diet prescribed to the individual assists in a reduction in weight of the individual. If the analysis is performed using the opposite DNA strand, the allele associated with this embodiment naturally comprises a “G” allele.

In one embodiment of this aspect and all other aspects disclosed herein, the method further comprises a step of first determining the individual's genotype at said perilipin locus rs2304795.

Another aspect described herein is a method to assist in decreasing body fat composition in an obese or overweight individual carrying at least one “C” allele at perilipin locus rs2304795, the method comprising the step of prescribing or advising the individual to follow a low carbohydrate diet to an individual carrying at least one “C” allele at perilipin locus rs2304795, wherein the low carbohydrate diet prescribed to the individual assists in decreasing body fat composition of the individual. If the analysis is performed using the opposite DNA strand, the allele associated with this embodiment naturally comprises a “G” allele.

In one embodiment of this aspect and all other aspects disclosed herein, the method further comprises a step of first determining the individual's genotype at the perilipin locus rs2304795.

Another aspect disclosed herein is a method to assist in improving metabolic rate in an overweight or obese individual carrying at least one “C” allele at perilipin locus rs2304795, the method comprising the step of prescribing or advising the individual to follow a low carbohydrate diet to an individual carrying at least one “C” allele at perilipin locus rs2304795, wherein the low carbohydrate diet prescribed to the individual assists in improving metabolic rate of the individual. If the analysis is performed using the opposite DNA strand, the allele associated with this embodiment naturally comprises a “G” allele.

In one embodiment of this aspect and all other aspects disclosed herein, the method further comprises a step of first determining the individual's genotype at the perilipin locus rs2304795.

Another aspect disclosed herein is a method for determining an optimal weight management program for an obese or overweight individual carrying at least one “T” allele at perilipin locus rs1052700, the method comprising the step of prescribing or advising the individual to follow a high complex carbohydrate diet to an individual carrying at least one “T” allele at perilipin locus rs1052700. If the analysis is performed using the opposite DNA strand, the allele associated with this embodiment naturally comprises an “A” allele.

In one embodiment of this aspect and all other aspects disclosed herein, the method further comprises a step of first determining the individual's genotype at the perilipin locus rs1052700.

Another aspect disclosed herein is a method for assisting in reducing weight in an overweight or obese individual carrying at least one “T” allele at perilipin locus rs1052700, the method comprising the step of prescribing or advising the individual to follow a high complex carbohydrate diet to the individual, wherein the high complex carbohydrate diet prescribed to the individual assists in a reduction in weight of the individual. If the analysis is performed using the opposite DNA strand, the allele associated with this embodiment naturally comprises an “A” allele.

In one embodiment of this aspect and all other aspects disclosed herein, the method further comprises a step of first determining the individual's genotype at said perilipin locus rs1052700.

Another aspect disclosed herein is a method to assist in decreasing body fat composition in an obese or overweight individual carrying at least one “T” allele at perilipin locus rs1052700, the method comprising the step of prescribing or advising the individual to follow a high complex carbohydrate diet to an individual carrying at least one “T” allele at perilipin locus rs1052700, wherein the high complex carbohydrate diet prescribed to the individual assists in decreasing body fat composition of the individual. If the analysis is performed using the opposite DNA strand, the allele associated with this embodiment naturally comprises an “A” allele.

In one embodiment of this aspect and all other aspects disclosed herein, the method further comprises a step of first determining the individual's genotype at the perilipin locus rs1052700.

Another aspect disclosed herein is a method for determining an optimal weight management program for an obese or overweight individual carrying at least one “A” allele at perilipin locus rs894160, the method comprising the step of prescribing or advising the individual to follow a high glycemic diet (i.e. low fat) to the individual carrying at least one “A” allele at perilipin locus rs894160. If the analysis is performed using the opposite DNA strand, the allele associated with this embodiment naturally comprises a “T” allele.

In one embodiment of this aspect and all other aspects disclosed herein, the method further comprises a step of first determining the individual's genotype at the perilipin locus rs894160.

Another aspect described herein is a method for assisting in reducing weight in an overweight or obese individual carrying at least one “G” allele at perilipin locus rs2289487, the method comprising the step of prescribing or advising the individual to follow a low carbohydrate diet to an individual carrying at least one “G” allele at perilipin locus rs2289487, wherein the high carbohydrate diet (i.e. low fat) prescribed to the individual assists in a reduction in weight of the individual. If the analysis is performed using the opposite DNA strand, the allele associated with this embodiment naturally comprises a “C” allele.

In one embodiment of this aspect and all other aspects disclosed herein, the method further comprises a step of first determining the individual's genotype at the perilipin locus rs2289487.

Also described herein is a method to assist in decreasing body fat composition in an obese or overweight individual carrying at least one “G” allele at perilipin locus rs2289487, the method comprising the step of prescribing or advising the individual to follow a low carbohydrate diet to the individual carrying at least one “G” allele at perilipin locus rs2289487, wherein the high carbohydrate diet (i.e. low fat) prescribed to the individual assists in decreasing body fat composition of the individual. If the analysis is performed using the opposite DNA strand, the allele associated with this embodiment naturally comprises a “C” allele.

In one embodiment of this aspect and all other aspects disclosed herein, the method further comprises a step of first determining the individual's genotype at the perilipin locus rs2289487.

Also described herein is a method to assist in improving metabolic rate in an overweight or obese individual carrying at least one “G” allele at perilipin locus rs2289487, the method comprising the step of prescribing or advising the individual to follow a high carbohydrate diet (i.e. low fat) to an individual carrying at least one “G” allele at perilipin locus rs2289487, wherein the low carbohydrate diet prescribed to the individual assists in improving metabolic rate of the individual. If the analysis is performed using the opposite DNA strand, the allele associated with this embodiment naturally comprises a “C” allele.

In one embodiment of this aspect and all other aspects described herein, the method further comprises a step of first determining the individual's genotype at the perilipin locus rs2289487.

According to some embodiments, kits are provided for determining a subject's response to low calorie or liquid diet toward achieving weight loss comprising reagents and instructions for genotyping said subject at one or more alleles at: SNP rs894160 of PLIN 4, SNP rs8179043 of PLIN Z, SNP rs2289487 of PLIN 1, SNP rs4578621 of PLIN X, and SNP rs1052700 of PLIN 6; wherein the presence of one or more alleles is predictive of said subject's predisposition to weight loss in response to low calorie diet, or liquid diet, or both.

According to some embodiments, kits are provided for determining a subject's response to low calorie or liquid diet toward achieving weight loss, comprising reagents and instructions for detecting in said subject an allele A at SNP rs894160 of PLIN 4, wherein the reagents comprises primers, buffers, salts for detecting said allele.

According to some embodiments, kits are provided for determining a subject's response to low calorie or liquid diet toward achieving weight loss, comprising reagents and instructions for detecting in said subject an allele A at SNP rs8179043 of PLIN Z, wherein the reagents comprises primers, buffers, salts for detecting said allele.

According to some embodiments, kits are provided for determining a subject's response to low calorie or liquid diet toward achieving weight loss, comprising reagents and instructions for detecting in said subject an allele G at SNP rs2289487 of PLIN 1, wherein the reagents comprises primers, buffers, salts for detecting said allele.

According to some embodiments, kits are provided for determining a subject's response to low calorie or liquid diet toward achieving weight loss comprising reagents and instructions for genotyping said subject for composite genotype at one at one or more alleles selected from the group consisting of: SNP rs894160 of PLIN 4, SNP rs8179043 of PLIN Z, SNP rs2289487 of PLIN 1, SNP rs4578621 of PLIN X, and SNP rs1052700 of PLIN 6; wherein the presence of one or more alleles is predictive of said subject's predisposition to weight loss in response to low calorie diet, or liquid diet, or both.

According to some embodiments, kits are provided for determining a subject's composite genotype, comprising reagents and instructions for: a) genotyping said subject at: (i) SNP rs894160 of PLIN 4; (ii) SNP rs8179043 of PLIN Z; and (iii) SNP rs2289487 of PLIN 1; b) determining whether said subject has a composite genotype comprising the allelic pattern or haplotype of: heretozygous allele A at SNP rs894160 of PLIN 4, heterozygous allele A at SNP rs8179043 of PLIN Z, and heterozygous allele G at SNP rs2289487 of PLIN 1; wherein the presence of the haplotype indicates said subject is resistant to weight loss in response to a low calorie or liquid diet.

According to some embodiments, kits are provided for determining a subject's composite genotype, comprising reagents and instructions for: a) genotyping said subject at: (i) SNP rs894160 of PLIN 4; (ii) SNP rs8179043 of PLIN Z; and (iii) SNP rs2289487 of PLIN 1; b) determining whether said subject has a composite genotype comprising the allelic pattern or haplotype of: heterozygous allele G at SNP rs894160 of PLIN 4, heterozygous allele G at SNP rs8179043 of PLIN Z, and heterozygous allele A at SNP rs2289487 of PLIN 1; wherein the presence of the haplotype indicates said subject is resistant to weight loss in response to a low calorie or liquid diet.

According to some embodiments, kits are provided for determining a subject's composite genotype, comprising reagents and instructions for: a) genotyping said subject at: (i) SNP rs894160 of PLIN 4; (ii) SNP rs8179043 of PLIN Z; (iii) SNP rs2289487 of PLIN 1; and (iv) SNP rs4578621 of PLIN X; b) determining whether said subject has a composite genotype comprising the allelic pattern or haplotype of: allele G at SNP rs894160 of PLIN 4, allele G at SNP rs8179043 of PLIN Z, allele A at SNP rs2289487 of PLIN 1 and allele G at SNP rs4578621 of PLIN X; wherein the presence of the haplotype indicates said subject is resistant to weight loss in response to a low calorie or liquid diet.

According to some embodiments, kits are provided for determining a subject's composite genotype, comprising reagents and instructions for: a) genotyping said subject at: (i) SNP rs894160 of PLIN 4; (ii) SNP rs8179043 of PLIN Z; (iii) SNP rs2289487 of PLIN 1; and (iv) SNP rs4578621 of PLIN X; b) determining whether said subject has a composite genotype comprising the allelic pattern or haplotype of: allele A at SNP rs894160 of PLIN 4, allele A at SNP rs8179043 of PLIN Z, allele G at SNP rs2289487 of PLIN 1 and allele G at SNP rs4578621 of PLIN X; wherein the presence of the haplotype indicates said subject is resistant to weight loss in response to a low calorie or liquid diet.

According to some embodiments, kits are provided for determining a subject's composite genotype, comprising reagents and instructions, wherein the reagents comprises primers, buffers, salts for detecting said composite genotype.

Nutrition Categories

The Geisinger Study was performed in two main stages. The stages were identified based on the numbers of calories consumed and the macronutrient composition. In stage 1 (subjects were placed on a “low calorie diet” for about 4 months), enrolled women were recommended a diet of 1100-1800 kcal. In some embodiments, women were provided a diet of 1700-1800 kcal, or 1600-1700 kcal, or 1500-1600 kcal or 1400-1500 kcal or 1300-1400 kcal or 1200-1300 kcal or 1100-1200 kcal. In some embodiments, women were provided a diet of 1200-1500 kcal, or 1100-1500 kcal, or 1500-1800 kcal. In a preferred embodiment, women were provided a diet of 1200 kcal.

In stage 1, men were provided with a low calorie diet in the range of 1400-2200 kcal. In some embodiments, men were provided a diet of 2100-2200 kcal, or 2000-2100 kcal, or 1900-2000 kcal, or 1800-1900 kcal, or 1700-1800 kcal, or 1600-1700 kcal, or 1500-1600 kcal, or 1400-1500 kcal. In some embodiments, men were provided a diet of 1500-1800 kcal, or 1400-1800 kcal, or 1800-2200 kcal, or 1600-2000 kcal. In a preferred embodiment, men were provided a diet of 1800 kcal.

In stage 2 (subjects were placed on a very low calorie “liquid diet” that replaced meals, were high in protein, and dramatically reduced both saturated fat and total fat, while also reducing carbohydrate intake for 120 days), enrolled women were recommended a diet of 800-1200 kcal. In some embodiments, women were provided a diet of 1100-1200 kcal, or 1000-1100 kcal, or 900-1000 kcal, or 800-900 kcal, or 900-1100 kcal. In a preferred embodiment, women were provided a diet of 1000 kcal per day.

In stage 2 (subjects were subjected to low calorie “liquid diet” for 120 days), enrolled men were recommended a diet of 1000-1500 kcal. In some embodiments, men were provided a diet of 1400-1500 kcal, or 1300-1400 kcal, or 1200-1300 kcal, or 1100-1200 kcal, or 1000-1100, or 1100-1300 kcal. In a preferred embodiment, men were provided a diet of 1200 kcal per day.

Subjects, who lost >3% weight after being on a recommended diet in stage 1, were classified as Group A. In stage 2 (after the first 4 months, another about 4 months) all subjects who lost <3% weight in stage 1 were recommended a liquid diet of 1000 kcal (women) or 1200 kcal (men). Once on liquid diet, subjects who lost >5% of total body weight in an early stage were classified as Group B (early responders), and those who lost the same amount of weight, but at a later stage were categorized in Group C (late responders). Subjects, who did not respond to either stages (I or II), were classified as Group D (non-responders).

According to some embodiments, the Group B early responders responded to liquid diet between 20-30 days, or 31-40 days, or 41-50 days, or 51-60 days, or 61-70 days, or 71-80 days, or 81-90 days, or 91-100 days, or 101-110 days, or 111-120 days. In some preferred embodiments, the Group B early responders responded between 20-120 days, or 20-60 days, or 30-60 days, or 30-120 days, or 60-120 days.

According to some embodiments, the Group C late responders responded to liquid diet between 120-130 days, or 131-140 days, or 141-150 days, or 151-160 days, or 161-170 days, or 171-180 days, or 181-190 days, or 191-200 days, or 201-210 days, or 211-220 days, or 221-230 days, or 231-240 days, or 241-250 days, or 251-260 days, or 261-270 days, or 271-280 days, or 281-290 days, or 291-300 days, or 301-310 days, or 311-320 days, or 321-330 days, or 331-340 days, or 341-350 days, or 351-360 days, or 361-370 days. In some preferred embodiments, the Group C late responders respond between 121-190 days, or 121-360 days, or 121-370 days, or 121-180 days, or 121-220 days, or 121-160 days, or 160-200 days, or 160-180 days, or 160-220 days, or 180-220 days, or 180-370 days.

Nutrition categories are generally classified on the basis of the amount of macronutrients (i.e., fat, carbohydrates, protein) recommended for a subject based on that subject's metabolic genotype. The primary goal of selecting an appropriate therapeutic/dietary regimen or lifestyle recommendation for a subject is to pair a subject's metabolic genotype with the nutrition category to which that subject is most likely to be responsive. A nutrition category is generally expressed in terms of the relative amounts of macronutrients suggested for a subject's diet or in terms of calories restrictions (e.g., restricting the total number of calories a subject receives and/or restricting the number of calories a subject receives from a particular macronutrient). For example, nutrition categories may include, but are not limited to, 1) high protein, low fat, low carbohydrate diets; 2) low fat diets, or 3) low carbohydrate diets. Alternatively, nutrition categories may be classified on the basis of the restrictiveness of certain macronutrients recommended for a subject based on that subject's metabolic genotype. For example, nutrition categories may be expressed as 1) balanced or calorie restricted diets; 2) fat restrictive diets, or 3) carbohydrate restrictive diets.

Subjects with a metabolic genotype that is responsive to fat restriction or low fat diet tend to absorb more dietary fat into the body and have a slower metabolism. They have a greater tendency for weight gain. Clinical studies have shown these subjects have an easier time reaching a healthy body weight by decreasing total dietary fat. They may have greater success losing weight by following a reduced fat and/or reduced calorie diet. In addition, they benefit from replacing saturated fats with monounsaturated fats within a reduced calorie diet. Clinical studies have also shown these same dietary modifications improve the body's ability to metabolize sugars and fats.

Subjects with a metabolic genotype that is responsive to carbohydrate restriction or low carbohydrate diet tend to be more sensitive to weight gain from excessive carbohydrate intake. They may have greater success losing weight by reducing carbohydrates within a reduced calorie diet. Subjects with this genetic pattern are prone to obesity and have difficulty with blood sugar regulation if their daily carbohydrate intake is high, such as where the daily carbohydrate intake exceeds, for example, about 49% of total calories. Carbohydrate reduction has been shown to optimize blood sugar regulation and reduce risk of further weight gain. If they have high saturated and low monounsaturated fats in their diet, risk for weight gain and elevated blood sugar increases. While limiting total calories, these subjects may benefit from restricting total carbohydrate intake and shifting the fat composition of their diet to monounsaturated fats (e.g., a diet low in saturated fat and low in carbohydrate).

Subjects with a metabolic genotype that is responsive to a balance of fat and carbohydrate show no consistent need for a low fat or low carbohydrate diet. In these subjects key biomarkers, such as body weight, body fat, and plasma lipid profile, respond well to a diet balanced in fat and carbohydrate. For subjects with this genetic pattern who are interested in losing weight, a balanced diet restricted in calories has been found to promote weight loss and a decrease in body fat, wherein the fat content of a subject is reduced irrespective of the body weight (lean body mass). Body fat may be measured by methods well known in the art. A preferred method is DEXA (Dual Energy X-ray Absorptiometry)—a technology that is very accurate and precise. DEXA is based on a three-compartment model that divides the body into total body mineral, fat-free soft (lean) mass, and fat tissue mass. This technique is based on the assumption that bone mineral content is directly proportional to the amount of photon energy absorbed by the bone being studied. Other methods for measurement of body fat includes, but not limited to: NIR (Near Infrared Interactance); MRI (Magnetic Resonance Imaging); TOBEC (Total Body Electrical Conductivity); CT (Compound Tomography); BOD POD (Air Displacement); BIA (Bioelectric al Impedance).

A low fat diet refers to a diet that provides between about 10% to less than about 30% of total calories from fat. According to some embodiments, a low fat diet refers to a diet that provides no more than about 30 percent (e.g., no more than about 19%, 21%, 23%, 22%, 24%, 26%, 28%, etc) of total calories from fat. According to some embodiments, a low fat diet refers to a diet that provides no more than about 30 percent of total calories from fat. According to some embodiments, a low fat diet refers to a diet that provides no more than about 25 percent of total calories from fat. According to some embodiments, a low fat diet refers to a diet that provides no more than about 20 percent of total calories from fat. According to some embodiments, a low fat diet refers to a diet that provides no more than about 15 percent of total calories from fat. According to some embodiments, a low fat diet refers to a diet that provides no more than about 10 percent of total calories from fat.

According to some embodiments, a low fat diet refers to a diet that is between about 10 grams and about 60 grams of fat per day. According to some embodiments, a low fat diet refers to a diet that is less than about 50 grams (e.g., less than about 10, 25, 35, 45, etc) grams of fat per day. According to some embodiments, a low fat diet refers to a diet that is less than about 40 grams of fat per day. According to some embodiments, a low fat diet refers to a diet that is less than about 30 grams of fat per day. According to some embodiments, a low fat diet refers to a diet that is less than about 20 grams of fat per day.

Fats contain both saturated and unsaturated (monounsaturated and polyunsaturated) fatty acids. According to some embodiments, reducing saturated fat to less than 10 percent of calories is a diet low in saturated fat. According to some embodiments, reducing saturated fat to less than 15 percent of calories is a diet low in saturated fat. According to some embodiments, reducing saturated fat to less than 20 percent of calories is a diet low in saturated fat.

A low carbohydrate (CHO) diet refers to a diet that provides between about 20% to less than about 50% of total calories from carbohydrates. According to some embodiments, a low carbohydrate (CHO) diet refers to a diet that provides no more than about 50 percent (e.g., no more than about 20%, 25%, 30%, 35%, 40%, 45%, etc) of total calories from carbohydrates. According to some embodiments, a low carbohydrate diet refers to a diet that provides no more than about 45 percent of total calories from carbohydrates. According to some embodiments, a low carbohydrate diet refers to a diet that provides no more than about 40 percent of total calories from carbohydrates. According to some embodiments, a low carbohydrate diet refers to a diet that provides no more than about 35 percent of total calories from carbohydrates. According to some embodiments, a low carbohydrate diet refers to a diet that provides no more than about 30 percent of total calories from carbohydrates. According to some embodiments, a low carbohydrate diet refers to a diet that provides no more than about 25 percent of total calories from carbohydrates. According to some embodiments, a low carbohydrate diet refers to a diet that provides no more than about 20 percent of total calories from carbohydrates.

A low carbohydrate (CHO) diet may refer to a diet that restricts the amount of grams of carbohydrate in a diet such as a diet of from about 20 to about 250 grams of carbohydrates per day. According to some embodiments, a low carbohydrate diet comprises no more than about 220 (e.g., no more than about 40, 70, 90, 110, 130, 180, 210, etc) grams of carbohydrates per day. According to some embodiments, a low carbohydrate diet comprises no more than about 200 grams of carbohydrates per day. According to some embodiments, a low carbohydrate diet comprises no more than about 180 grams of carbohydrates per day. According to some embodiments, a low carbohydrate diet comprises no more than about 150 grams of carbohydrates per day. According to some embodiments, a low carbohydrate diet comprises no more than about 130 grams of carbohydrates per day. According to some embodiments, a low carbohydrate diet comprises no more than about 100 grams of carbohydrates per day. According to some embodiments, a low carbohydrate diet comprises no more than about 75 grams of carbohydrates per day.

A low carbohydrate diet may also be referred to as a low-glycemic-load diet, and a high carbohydrate diet may also be referred to as high-glycemic-load diet. According to some embodiments, a high-glycemic (HG or high CHO) diet and a low-glycemic (LG or low CHO) diet may both be designed to promote calorie restriction (CR), while differing in the ratio of macronutrients. That is, the diets may differ in the ratio of macronutrients (for example, HG: 60% carbohydrate, 20% fat, and 20% protein; and LG: 40% carbohydrate, 30% fat, and 30% protein). The carbohydrate sources in the LG diet preferably have a lower glycemic index (GI) per published GIs of different carbohydrate sources (see e.g., International table of glycemic index and glycemic load values: 2002. Am J Clin Nutr 2002; 76:5-56, incorporated herein by reference in its entirety).

Examples of food used for a HG diet include, but are not limited to, the following: candied sweet potatoes; carrots; chicken and pea casserole; chef salad; chicken and rice; couscous; english muffins and bagels; jelly; jasmine rice; Lactose-free skim milk (Lactaid; McNeil Nutritionals, LLC, Fort Washington, Pa.); oatmeal; pizza; sugar cookies and graham crackers; shepherd's pie with mashed potatoes; sweet and sour chicken; turkey with cranberry sauce; tuna sandwich; waffles; and yogurt with added fruit—canned pears, peaches, figs, pineapple, oranges, and bananas. Examples of food used for a LG diet include, but are not limited to, the following: baked chicken; bean and barley stew; bulgur and beans; broccoli and beans; cottage cheese, low-fat; curried lentils; fish; fruit: oranges, grapefruit, plums, pears, apples, and berries; flaxseed cookies; green salad; Kashi (Kashi, La Jolla, Calif.) and Muesli cereal (Kellogg's Co, Battle Creek, Mich.); lentils with tomato sauce; nuts; pumpernickel bread; salisbury steak; skim milk; tomato cucumber bean salad; wheat berry salad; and yogurt.

According to some embodiments, both the HG and LG diets may be designed with features to promote calorie restriction, including, but not limited to, the following: meeting Dietary Reference Intakes (DRIs) for dietary fiber (Institute of Medicine. Dietary reference intakes: energy, carbohydrate, fiber, fat, fatty acids, cholesterol, protein, and amino acids. Vol 5. Washington, D.C.: The National Academy Press, 2002:1-114, incorporated herein by reference in its entirety); limited inclusion of high-energy-density foods (as defined in Rolls et al., J Am Diet Assoc 2005; 105(suppl):S98-103, incorporated herein by reference in its entirety); limited liquid calories (as defined in Mattes, Physiol Behav 1996; 59: 179-87, incorporated herein by reference in its entirety); and a relatively high variety of low-energy-density foods (e.g., fruit and vegetables), and a relatively low variety of high-energy-dense foods (as defined in McCrory et al., Am J Clin Nutr 1999; 69:440-7, incorporated herein by reference in its entirety).

A calorie restricted (CR) diet or balanced diet refers to a diet that restricts total calories consumed to below a subject's weight maintenance level (WML), regardless of any preference for a macronutrient. A balanced diet or calorie restricted diet seeks to reduce the overall caloric intake of a subject by, for example, reducing the total caloric intake of a subject to below that subject's WML without a particular focus on restricting the calories consumed from any particular macronutrient. For example, calorie restricted diet may contain the range of current dietary recommendations for healthful macronutrient ranges and containing the Dietary Reference Intakes (DRIs) of micronutrients and essential fatty acids at 10-50% (e.g., 10% 15%, 20%, 25%, 30%, 35%, 40%, 45%, or 50%) calorie restriction (CR) relative to baseline energy requirements. Thus, according to some embodiments, a balanced diet may be expressed as a percentage of a subject's WML. For example, a balanced diet is a diet that comprises a total caloric intake of between about 50% to about 100% WML. According to some embodiments, a balanced diet is a diet that comprises a total caloric intake of less than 100% (e.g., less than about 99%, 97%, 95%, 90%, 85%, 80%, 75%, 70%, 65%, 60%, 55%) of WML. Within this framework, a balanced diet achieves a healthy or desired balance of macronutrients in the diet and may be: low fat; low saturated fat; low carbohydrate; low fat and low carbohydrate; or low saturated fat and low carbohydrate. For example, a diet may be a low fat, calorie restricted diet (where low fat has the meaning as provided hereinabove). A diet may be a low carbohydrate, calorie restricted diet (where low carbohydrate has the meaning as provided hereinabove). A diet may be a balanced, calorie restricted diet (e.g., relative portions of macronutrients may vary where the total calories consumed is below the WML).

According to some embodiments, a low-carb diet (Carb: 45%, Protein: 20%, and Fat: 35%) comprises any of: Atkins diet, Glycemic Impact Diet, South Beach Diet, Sugar Busters Diet, and/or Zone diet.

According to some embodiments, a low-fat diet (Carb: 65%, Protein: 15%, Fat: 20%) comprises any of: Life Choice Diet (Ornish Diet), Pritikin Diet, and/or other heart healthy diets available in the market.

According to some embodiments, a balanced diet (Carb: 55%, Protein: 20%, Fat: 25%) comprises any of: Best Life Diet, Mediterranean Diet, Sonoma Diet, Volumetrics Eating Diet, Weight Watchers Diet.

Other low carbohydrate, low fat, balanced diet or calorie restricted diets are well known in the art, thus can be recommended to a subject depending on the subject's metabolic genotype and predicted response to calorie restricted or other diet types.

In addition to the nutritional and exercise recommendations, the personalized therapeutic/dietary regimen may also include recommendation for dietary supplements, food supplements, or nutraceuticals. A “nutraceutical” is any functional food that provides an additional benefit other than its nutritional benefit. This category may include nutritional drinks, diet drinks (e.g., Slimfast™ and the like) as well as sports herbal and other fortified beverages.

Assessing Efficacy of a Dietary Treatment

The efficacy of dietary treatment can be determined by a skilled clinician. However, a treatment is considered “effective,” as the term is used herein, if any one or all of the signs or symptoms of obesity are altered in a beneficial manner, other clinically accepted symptoms are improved, or even ameliorated, e.g., by at least 10% following treatment with a diet and/or an anti-obesity agent. Efficacy can also be measured by a failure of an obese individual to develop an obesity-related disease or get worse (i.e., progression of obesity is halted). Methods of measuring these indicators are known to those of skill in the art and/or are described herein.

Treatment includes any treatment of a disease in a human individual and includes: (1) preventing the disease from occurring in an individual which may be predisposed to the disease but does not yet experience or display symptoms of the disease; e.g., prevention of obesity in an individual having lost at least one BMI point; (2) inhibiting weight gain or (3) inducing weight loss. An effective diet for the treatment of a disease means that diet which, when administered to an individual in need thereof, is sufficient to result in effective treatment as that term is defined herein, for that disease.

Efficacy of a particular diet can be determined by assessing physical indicators of obesity, such as e.g., BMI, waist-to-hip ratio, body fat percentage, total weight loss, percent weight loss, systolic blood pressure, diastolic blood pressure, and waist circumference. In general, a physical parameter (or set of parameters) will be measured in an individual prior to the onset of treatment. The same physical parameter or set thereof, is measured at various time points and compared to the original value of the measured parameter for that individual. A reduction in BMI, waist-to-hip ratio, body fat percentage, total weight or waist circumference are indicators that a diet is efficacious for that individual.

Efficacy of the diet can also be determined by assessing metabolic effects associated with obesity, including e.g., fasting blood glucose, serum triglycerides, insulin, high-density lipoprotein cholesterol, or low-density lipoprotein cholesterol. Metabolic effects can be measured by obtaining e.g., a blood sample from an individual being treated for obesity. A reduction in fasting blood glucose, total serum triglycerides, insulin, or low-density lipoprotein cholesterol indicates that an agent is efficacious at the current dose regime. Conversely, an increase in the level of high-density lipoprotein cholesterol indicates a normalization of metabolic effects due to obesity.

Detection of Alleles

Allelic patterns, polymorphism patterns, or haplotype patterns can be identified by detecting any of the component alleles using any of a variety of available techniques, including: 1) performing a hybridization reaction between a nucleic acid sample and a probe that is capable of hybridizing to the allele; 2) sequencing at least a portion of the allele; or 3) determining the electrophoretic mobility of the allele or fragments thereof (e.g., fragments generated by endonuclease digestion). The allele can optionally be subjected to an amplification step prior to performance of the detection step. Preferred amplification methods are selected from the group consisting of: the polymerase chain reaction (PCR), the ligase chain reaction (LCR), strand displacement amplification (SDA), cloning, and variations of the above (e.g. RT-PCR and allele specific amplification). Oligonucleotides necessary for amplification may be selected, for example, from within the metabolic gene loci, either flanking the marker of interest (as required for PCR amplification) or directly overlapping the marker (as in allele specific oligonucleotide (ASO) hybridization). In a particularly preferred embodiment, the sample is hybridized with a set of primers, which hybridize 5′ and 3′ in a sense or antisense sequence to the vascular disease associated allele, and is subjected to a PCR amplification.

An allele may also be detected indirectly, e.g. by analyzing the protein product encoded by the DNA. For example, where the marker in question results in the translation of a mutant protein, the protein can be detected by any of a variety of protein detection methods. Such methods include immunodetection and biochemical tests, such as size fractionation, where the protein has a change in apparent molecular weight either through truncation, elongation, altered folding or altered post-translational modifications.

A general guideline for designing primers for amplification of unique human chromosomal genomic sequences is that they possess a melting temperature of at least about 50° C., wherein an approximate melting temperature can be estimated using the formula Tₘₑₗₜ=[2X(# of A or T)+4X(# of G or C)].

Many methods are available for detecting specific alleles at human polymorphic loci. The preferred method for detecting a specific polymorphic allele will depend, in part, upon the molecular nature of the polymorphism. For example, the various allelic forms of the polymorphic locus may differ by a single base-pair of the DNA. Such single nucleotide polymorphisms (or SNPs) are major contributors to genetic variation, comprising some 80% of all known polymorphisms, and their density in the human genome is estimated to be on average 1 per 1,000 base pairs. SNPs are most frequently biallelic-occurring in only two different forms (although up to four different forms of an SNP, corresponding to the four different nucleotide bases occurring in DNA, are theoretically possible). Nevertheless, SNPs are mutationally more stable than other polymorphisms, making them suitable for association studies in which linkage disequilibrium between markers and an unknown variant is used to map disease-causing mutations. In addition, because SNPs typically have only two alleles, they can be genotyped by a simple plus/minus assay rather than a length measurement, making them more amenable to automation.

A variety of methods are available for detecting the presence of a particular single nucleotide polymorphic allele in a subject. Advancements in this field have provided accurate, easy, and inexpensive large-scale SNP genotyping. Most recently, for example, several new techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods require amplification of the target genetic region, typically by PCR. Still other newly developed methods, based on the generation of small signal molecules by invasive cleavage followed by mass spectrometry or immobilized padlock probes and rolling-circle amplification, might eventually eliminate the need for PCR. Several of the methods known in the art for detecting specific single nucleotide polymorphisms are summarized below. The method of the present invention is understood to include all available methods.

Several methods have been developed to facilitate analysis of single nucleotide polymorphisms. In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Publication No. WO92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Publication No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

For mutations that produce premature termination of protein translation, the protein truncation test (PTT) offers an efficient diagnostic approach (Roest, et. al., (1993) Hum. Mol. Genet. 2:1719-21; van der Luijt, et. al., (1994) Genomics 20:1-4). For PTT, RNA is initially isolated from available tissue and reverse-transcribed, and the segment of interest is amplified by PCR. The products of reverse transcription PCR are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products. Upon sodium dodecyl sulfate-polyacrylamide gel electrophoresis of translation products, the appearance of truncated polypeptides signals the presence of a mutation that causes premature termination of translation. In a variation of this technique, DNA (as opposed to RNA) is used as a PCR template when the target region of interest is derived from a single exon.

Any cell type or tissue may be utilized to obtain nucleic acid samples for use in the diagnostics described herein. In a preferred embodiment, the DNA sample is obtained from a bodily fluid, e.g, blood, obtained by known techniques (e.g. venipuncture) or saliva. Alternatively, nucleic acid tests can be performed on dry samples (e.g. hair or skin). When using RNA or protein, the cells or tissues that may be utilized must express a metabolic gene of interest.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

A preferred detection method is allele specific hybridization using probes overlapping a region of at least one allele of a metabolic gene or haplotype and having about 5, 10, 20, 25, or 30 nucleotides around the mutation or polymorphic region. In a preferred embodiment of the invention, several probes capable of hybridizing specifically to other allelic variants of key metabolic genes are attached to a solid phase support, e.g., a “chip” (which can hold up to about 250,000 oligonucleotides). Oligonucleotides can be bound to a solid support by a variety of processes, including lithography. Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

These techniques may also comprise the step of amplifying the nucleic acid before analysis. Amplification techniques are known to those of skill in the art and include, but are not limited to cloning, polymerase chain reaction (PCR), polymerase chain reaction of specific alleles (ASA), ligase chain reaction (LCR), nested polymerase chain reaction, self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), and Q-Beta Replicase (Lizardi, P. M. et al., 1988, Bio/Technology 6:1197).

Amplification products may be assayed in a variety of ways, including size analysis, restriction digestion followed by size analysis, detecting specific tagged oligonucleotide primers in the reaction products, allele-specific oligonucleotide (ASO) hybridization, allele specific 5′ exonuclease detection, sequencing, hybridization, and the like.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize 5′ and 3′ to at least one allele of a metabolic gene or haplotype under conditions such that hybridization and amplification of the allele occurs, and (iv) detecting the amplification product. These detection schemes are especially useful for the detection of nucleic acid molecules if such molecules are present in very low numbers.

In a preferred embodiment of the subject assay, the allele of a metabolic gene or haplotype is identified by alterations in restriction enzyme cleavage patterns. For example, sample and control DNA is isolated, amplified (optionally), digested with one or more restriction endonucleases, and fragment length sizes are determined by gel electrophoresis.

In yet another embodiment, any of a variety—of sequencing reactions known in the art can be used to directly sequence the allele. Exemplary sequencing reactions include those based on techniques developed by Maxim and Gilbert ((1977) Proc. Natl. Acad Sci USA 74:560) or Sanger (Sanger et al (1977) Proc. Nat. Acad. Sci USA 74:5463). It is also contemplated that any of a variety of automated sequencing procedures may be utilized when performing the subject assays (see, for example Biotechniques (1995) 19:448), including sequencing by mass spectrometry (see, for example PCT publication WO 94/16101; Cohen et al. (1996) Adv Chromatogr 36:127-162; and Griffin et al. (1993) Appl Biochem Biotechnol 38:147-159). It will be evident to one of skill in the art that, for certain embodiments, the occurrence of only one, two or three of the nucleic acid bases need be determined in the sequencing reaction. For instance, A-track or the like, e.g., where only one nucleic acid is detected, can be carried out.

In a further embodiment, protection from cleavage agents (such as a nuclease, hydroxylamine or osmium tetroxide and with piperidine) can be used to detect mismatched bases in RNA/RNA or RNA/DNA or DNA/DNA heteroduplexes (Myers, et al. (1985) Science 230:1242). In general, the art technique of “mismatch cleavage” starts by providing heteroduplexes formed by hybridizing (labeled) RNA or DNA containing the wild-type allele with the sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with 51 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl. Acad Sci USA 85:4397; and Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an allele of a metabolic gene locus haplotype is hybridized to a CDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify a metabolic gene locus allele. For example, single strand conformation polymorphism (SSCP) may be used to detect differences in electrophoretic mobility between mutant and wild type nucleic acids (Orita et al. (1989) Proc Natl. Acad. Sci USA 86:2766, see also Cotton (1993) Mutat Res 285:125-144; and Hayashi (1992) Genet Anal Tech Appl 9:73-79). Single-stranded DNA fragments of sample and control metabolif locus alleles are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of alleles in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:12753).

Examples of other techniques for detecting alleles include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saiki et al. (1986) Nature 324:163); Saiki et al (1989) Proc. Natl. Acad. Sci USA 86:6230). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labelled target DNA.

Alternatively, allele specific amplification technology which depends on selective PCR amplification may be used in conjunction with the instant invention. Oligonucleotides used as primers for specific amplification may carry the mutation or polymorphic region of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 1 1:238). In addition it may be desirable to introduce a novel restriction site in the region of the mutation to create cleavage-based detection (Gasparini et al (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

In another embodiment, identification of the allelic variant is carried out using an oligonucleotide ligation assay (OLA), as described, e.g., in U.S. Pat. No. 4,998,617 and in Landegren, U. et al. ((1988) Science 241:1077-1080). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al. (1990) Proc. Natl. Acad. Sci. USA 87:8923-27). In this method, PCR is used to achieve the exponential amplification of target DNA, which is then detected using OLA.

Several techniques based on this OLA method have been developed and can be used to detect alleles of a metabolic gene locus haplotype. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in To be et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

Another embodiment of the invention is directed to kits for detecting a predisposition for responsiveness to certain diets and/or activity levels. This kit may contain one or more oligonucleotides, including 5′ and 3′ oligonucleotides that hybridize 5′ and 3′ to at least one allele of a metabolic gene locus or haplotype. PCR amplification oligonucleotides should hybridize between 25 and 2500 base pairs apart, preferably between about 100 and about 500 bases apart, in order to produce a PCR product of convenient size for subsequent analysis.

In another aspect, the invention features kits for performing the above-described assays. According to some embodiments, the kits of the present invention may include a means for determining a subject's genotype with respect to one or more metabolic gene. The kit may also contain a nucleic acid sample collection means. The kit may also contain a control sample either positive or negative or a standard and/or an algorithmic device for assessing the results and additional reagents and components including: DNA amplification reagents, DNA polymerase, nucleic acid amplification reagents, restrictive enzymes, buffers, a nucleic acid sampling device, DNA purification device, deoxynucleotides, oligonucleotides (e.g. probes and primers) etc.

For use in a kit, oligonucleotides may be any of a variety of natural and/or synthetic compositions such as synthetic oligonucleotides, restriction fragments, cDNAs, synthetic peptide nucleic acids (PNAs), and the like. The assay kit and method may also employ labeled oligonucleotides to allow ease of identification in the assays. Examples of labels which may be employed include radio-labels, enzymes, fluorescent compounds, streptavidin, avidin, biotin, magnetic moieties, metal binding moieties, antigen or antibody moieties, and the like.

As described above, the control may be a positive or negative control. Further, the control sample may contain the positive (or negative) products of the allele detection technique employed. For example, where the allele detection technique is PCR amplification, followed by size fractionation, the control sample may comprise DNA fragments of the appropriate size. Likewise, where the allele detection technique involves detection of a mutated protein, the control sample may comprise a sample of mutated protein. However, it is preferred that the control sample comprises the material to be tested. For example, the controls may be a sample of genomic DNA or a cloned portion of a metabolic gene. Preferably, however, the control sample is a highly purified sample of genomic DNA where the sample to be tested is genomic DNA.

The oligonucleotides present in said kit may be used for amplification of the region of interest or for direct allele specific oligonucleotide (ASO) hybridization to the markers in question. Thus, the oligonucleotides may either flank the marker of interest (as required for PCR amplification) or directly overlap the marker (as in ASO hybridization).

Information obtained using the assays and kits described herein (alone or in conjunction with information on another genetic defect or environmental factor, which contributes to osteoarthritis) is useful for determining whether a non-symptomatic subject has or is likely to develop the particular disease or condition. In addition, the information can allow a more customized approach to preventing the onset or progression of the disease or condition. For example, this information can enable a clinician to more effectively prescribe a therapy that will address the molecular basis of the disease or condition.

The kit may, optionally, also include DNA sampling means. DNA sampling means are well known to one of skill in the art and can include, but not be limited to substrates, such as filter papers, the AmpliCard™ (University of Sheffield, Sheffield, England S10 2JF; Tarlow, J W, et al., J. of Invest. Dermatol. 103:387-389 (1994)) and the like; DNA purification reagents such as Nucleon™ kits, lysis buffers, proteinase solutions and the like; PCR reagents, such as 10× reaction buffers, thernostable polymerase, dNTPs, and the like; and allele detection means such as the HinfI restriction enzyme, allele specific oligonucleotides, degenerate oligonucleotide primers for nested PCR from dried blood.

### DEFINITIONS

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting. Other features and advantages of the invention will be apparent from the following detailed description and claims.

For the purposes of promoting an understanding of the embodiments described herein, reference will be made to preferred embodiments and specific language will be used to describe the same. The terminology used herein is for the purpose of describing particular embodiments only, and is not intended to limit the scope of the present invention. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth.

The term “allele” refers to the different sequence variants found at different polymorphic regions. The sequence variants may be single or multiple base changes, including without limitation insertions, deletions, or substitutions, or may be a variable number of sequence repeats.

The term “allelic pattern” refers to the identity of an allele or alleles at one or more polymorphic regions. Alternatively, an allelic pattern may consist of either a homozygous or heterozygous state at a single polymorphic site. Alternatively, an allelic pattern may consist of the identity of alleles at more than one polymorphic site.

The terms “control” or “control sample” refer to any sample appropriate to the detection technique employed. The control sample may contain the products of the allele detection technique employed or the material to be tested. Further, the controls may be positive or negative controls. By way of example, where the allele detection technique is PCR amplification, followed by size fractionation, the control sample may comprise DNA fragments of an appropriate size. Likewise, where the allele detection technique involves detection of a mutated protein, the control sample may comprise a sample of a mutant protein. However, it is preferred that the control sample comprises the material to be tested. For example, the controls may be a sample of genomic DNA or a cloned portion containing one or more metabolic genes. However, where the sample to be tested is genomic DNA, the control sample is preferably a highly purified sample of genomic DNA.

Body mass index (BMI) is a measure of body fat based on height and weight that applies to both men and women. BMI is considered in to fall into the so called “normal” range when BMI is between 18.5-24.9. According to this invention, an underweight subject has a BMI<18.5; an overweight subject in the range 25-29.9 and an obese subject has a BMI of 30-39.9, and BMI of 40 or greater is considered extremely obese.

The term “comprising” or “comprises” is used to refer to compositions, methods, and respective component(s) thereof, that are essential to the invention, yet open to the inclusion of unspecified elements, whether essential or not. For example, the term comprises as used in “comprises a low glycemic diet” is used to refer to a weight management program that includes the low glycemic diet and may also include any other element or combination of elements useful for weight loss (e.g., increased exercise, number of meals, timing of meals, community support, nutritional guidance etc) as part of an optimal weight management program. As used herein the term “consisting essentially of” describes the incorporation of other elements that can be included in the description of the composition, method or respective component thereof and are limited to those that do not materially affect the basic and novel characteristic(s) of the invention. For example, the term “consisting essentially of” a low glycemic diet would describe a low glycemic diet wherein an individual is encouraged to exercise, and maintain their regular routines for food intake (i.e., timing of meals, number of meals etc). The term “consisting of” refers to inventions, compositions, methods, and respective components thereof as described herein, which are intended to be exclusive of any element not deemed an essential element to the component, composition or method. For example, the term “consisting of a low glycemic diet” suggests that the low glycemic diet is the only intervention recommended for a weight loss regime to obese or overweight individuals.

The phrases “disruption of the gene” and “targeted disruption” or any similar phrase refers to the site specific interruption of a native DNA sequence so as to prevent expression of that gene in the cell as compared to the wild-type copy of the gene. The interruption may be caused by deletions, insertions or modifications to the gene, or any combination thereof.

The term “haplotype” as used herein is intended to refer to a set of alleles that are inherited together as a group (are in linkage disequilibrium) at statistically significant levels (P_{corr}<0.05). As used herein, the phrase “metabolic haplotype” refers to a haplotype of metabolic gene loci.

“Increased risk” refers to a statistically higher frequency of occurrence of the disease or condition in a subject carrying a particular polymorphic allele in comparison to the frequency of occurrence of the disease or condition in a member of a population that does not carry the particular polymorphic allele.

The term “isolated” as used herein with respect to nucleic acids, such as DNA or RNA, refers to molecules separated from other DNAs, or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

The term “liquid diet” refers to a very low calorie meal replacement that is high in protein and very low in fats and carbohydrates.

The term “low glycemic diet” refers to a diet comprising carbohydrates that are broken down and absorbed into the bloodstream at a relatively slow rate (i.e., foods having a low glycemic index compared to that of glucose absorption). Conversely, the term “high glycemic diet” refers to a diet comprising carbohydrates that are broken down and absorbed into the bloodstream at a rate relatively faster (i.e., high glycemic index) than that of a low glycemic diet. The “glycemic index” of a food is generally defined by the area under the two hour blood glucose response curve (AUC) following the ingestion of a fixed portion of carbohydrate (usually 50 g). The AUC of the test food is divided by the AUC of the standard (i.e., glucose) and multiplied by 100. In general, the glycemic index of a food is determined by comparing the rate of dietary carbohydrate absorption to the rate of glucose absorption into the bloodstream. The glycemic index for glucose is used as a reference and is set at 100 units by definition. Foods having a glycemic index less than or equal to 55 units are considered to be “low glycemic”, while foods having a glycemic index within the range of 56-99 units are considered to be “high glycemic”. The glycemic content of a diet is dependent on many variables including, but not limited to the following: the proportion of fat, protein, fiber, organic acids or organic salts in the diet. A low glycemic diet is that in which the emphasis is placed on foods with low glycemic index (GI). Foods having a low GI are fruit and vegetables (except potatoes and watermelon), grainy breads, pasta, legumes, certain rice and milk. Foods having a medium GI are wheat bread, whole wheat products in general, brown rice, orange sweet potato, table sugar. Foods having a high GI include corn flakes, baked potato, some white rices (e.g., jasmine), croissant, white bread, candy. Usually a low glycemic diet will also be a low carbohydrate diet, but it could also be a high carbohydrate diet as long as the only carbohydrates consumed are those from the list of low GI foods.

“Linkage disequilibrium” refers to co-inheritance of two alleles at frequencies greater than would be expected from the separate frequencies of occurrence of each allele in a given control population. The expected frequency of occurrence of two alleles that are inherited independently is the frequency of the first allele multiplied by the frequency of the second allele. Alleles that co-occur at expected frequencies are said to be in “linkage disequilibrium”. The cause of linkage disequilibrium is often unclear. It can be due to selection for certain allele combinations or to recent admixture of genetically heterogeneous populations. In addition, in the case of markers that are very tightly linked to a disease gene, an association of an allele (or group of linked alleles) with the disease gene is expected if the disease mutation occurred in the recent past, so that sufficient time has not elapsed for equilibrium to be achieved through recombination events in the specific chromosomal region. When referring to allelic patterns that are comprised of more than one allele, a first allelic pattern is in linkage disequilibrium with a second allelic pattern if all the alleles that comprise the first allelic pattern are in linkage disequilibrium with at least one of the alleles of the second allelic pattern.

The term “marker” refers to a sequence in the genome that is known to vary among subjects.

A “mutated gene” or “mutation” or “functional mutation” refers to an allelic form of a gene, which is capable of altering the phenotype of a subject having the mutated gene relative to a subject which does not have the mutated gene. The altered phenotype caused by a mutation can be corrected or compensated for by certain agents. If a subject must be homozygous for this mutation to have an altered phenotype, the mutation is said to be recessive. If one copy of the mutated gene is sufficient to alter the phenotype of the subject, the mutation is said to be dominant. If a subject has one copy of the mutated gene and has a phenotype that is intermediate between that of a homozygous and that of a heterozygous subject (for that gene), the mutation is said to be co-dominant.

As used herein, the term “nucleic acid” refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs (e.g. peptide nucleic acids) and as applicable to the embodiment being described, single (sense or antisense) and double-stranded polynucleotides.

The phrase “optimal weight management program” is used to describe a diet plan comprising a proportion of carbohydrate, fat and protein in the total caloric amount that promotes the reduction of weight and/or the prevention of weight gain in an individual. Since not all obese or overweight individuals respond to a given diet, an “optimal weight management program” is designed to produce the largest amount of safe weight loss over time in an individual compared to the weight loss of that individual on a diet of similar caloric value but having varying proportions of carbohydrate, fat and protein. For the purposes of the methods of this invention, the optimal weight management program is determined by and dependent on an individual's genotype for a polymorphic marker at the perilipin locus.

The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene”. A specific genetic sequence at a polymorphic region of a gene is an allele. A polymorphic region can be a single nucleotide, the identity of which differs in different alleles. A polymorphic region can also be several nucleotides long.

The term “polymorphic marker” is used herein to describe a single nucleotide polymorphism (SNP), which is a DNA sequence variation occurring when a single nucleotide in the genome (or other shared sequence) differs between paired chromosomes in an individual or among individuals in a population.

The term “propensity to disease,” also “predisposition” or “susceptibility” to disease or any similar phrase, means that certain alleles are hereby discovered to be associated with or predictive of a subject's incidence of developing a particular disease (e.g. a vascular disease). The alleles are thus over-represented in frequency in subjects with disease as compared to healthy subjects. Thus, these alleles can be used to predict disease even in pre-symptomatic or pre-diseased subjects.

The term “preventing weight gain” as used herein describes a weight gain of an individual being treated with both an insulin sensitizing agent and an optimal weight management program that is less than 30% of the weight gain of that individual being treated with an insulin sensitizing agent but without a dietary intervention; preferably the weight gain of an individual on an optimal weight management program is less than 20%, less than 10%, less than 5% or even zero weight gain compared to the same individual not on an optimal weight management program. It is also contemplated herein that an individual being treated with both an insulin sensitizing agent an optimal weight management program may lose weight from the initial starting weight of that individual prior to beginning treatment with an optimal weight management program. For example, an individual may lose at least one BMI value, at least 2, at least 3, at least 4, at least 5, at least 10, at least 20, at least 30 BMI values or more, provided that the individual does not go below a BMI value of 18.5 wherein the individual would then be considered underweight and thus not having an optimal or healthy weight.

The phrase “responsiveness of an individual to caloric restriction” is used to denote the amount of weight loss or percent fat loss over time in individuals having a caloric intake of no less than 1500 calories per day (men) or no less than 1200 calories per day (women) compared to a control individual. Caloric restriction can also be determined by measuring the total energy expenditure of an individual as described by Das, S K et al., (2007) Am J Clin Nutr, 85:1023-30, which is incorporated herein by reference in its entirety. Briefly, total energy expenditure is determined by administering doubly labeled water (²H₂¹⁸O) and measuring abundance of H₂¹⁸O and ²H₂0 in urine specimens from a subject. The total energy expenditure is calculated as described by Das, S K, et al., (2007) and indicates the necessary amount of energy that an individual needs to maintain energy requirements while maintaining a constant weight. In general, a caloric restriction between at least 10-30% of the total energy expenditure will permit an individual to lose weight. Thus, the following formula can be used to determine the appropriate caloric intake for each individual: Total Energy Expenditure (kcal/d)×% CR=Energy intake on diet plan (kcal/day). As described by Das, S K., et al (2007), at the beginning of a diet plan subjects can be prescribed a 20-30% caloric restriction, however after some time on a caloric restricted diet one of skill in the art may reduce the caloric restriction to 10-15%, or lower in order to reduce the pace of weight loss and maintain individuals within a healthy weight range.

Not all individuals will respond, or respond well, by losing weight to a diet where the total caloric intake is decreased. The “responsiveness of an individual to caloric restriction” is not only dependent on the amount of caloric intake but also depends on an individual's genetic propensity for obesity and weight gain. The responsiveness of an individual to caloric restriction can be assessed by comparing an individual's weight loss on a calorie restricted diet to a control individual that is known to respond to the same calorie restricted diet. For the purposes of this application, a “control individual that is known to respond to a calorie restricted diet” is defined as one that loses weight or body fat when compliant with a calorie restricted diet for at least 3 months and/or having a wild-type genotype at the perilipin locus i.e., does not carry a rs2304795 (PLIN5) or rs1052700 (PLIN6) polymorphism (on either perilipin allele). An individual does not respond to caloric restriction if that individual does not lose at least 60% of the amount of weight lost by a wild-type individual on the same diet, preferably the individual does not lose at least 50%, at least 40%, at least 30%, at least 20%, at least 10%, at least 5% or less of the amount of weight lost by a wild-type individual on the same calorie restricted diet. In addition, an individual is considered to be a non-responder to caloric restriction if that individual does not lose any weight over a period of time exceeding at least 3 months during which the individual is compliant with a calorie restricted diet.

The phrase “reduction in weight of said individual” is used to denote a decrease in total body mass of an individual on a diet optimized for their genotype at the perilipin locus of at least 5 lbs compared to the body mass of that individual when not on diet optimized for their genotype at the perilipin locus. Preferably, the individual will lose at least 10 lbs, at least 15 lbs, at least 20 lbs, at least 25 lbs, at least 30 lbs, at least 35 lbs, at least 40 lbs, at least 50 lbs, at least 75 lbs, at least 100 lbs, at least 150 lbs, at least 200 lbs or more than on a calorie restricted diet that is not optimized to their genotype, provided that the individual does not go below a BMI value of 18.5.

As used herein, the term “specifically hybridizes” or “specifically detects” refers to the ability of a nucleic acid molecule to hybridize to at least approximately 6 consecutive nucleotides of a sample nucleic acid.

“Transcriptional regulatory sequence” is a generic term used throughout the specification to refer to DNA sequences, such as initiation signals, enhancers, and promoters, which induce or control transcription of protein coding sequences with which they are operably linked.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

The term “risk-allele” refers to an allele of a gene which, when present in one or two copies in a subject results in increased propensity to a disorder, or phenotype under investigation. There can be several different risk-alleles, since several different nucleotide changes in a gene may affect the phenotype under study, with a variation in intensity. The term “risk-allele,” thus refers to an SNP or allele that is associated with high relative risk for a disorder or phenotype under investigation.

Genotype Definitions

TABLE 1

| | | |
|---|---|---|
| PLIN 1 | A/A | 1.1 |
| rs2289487 | A/G | 1.2 |
| | G/G | 2.2 |
| PLIN 4 | G/G | 1.1 |
| rs894160 | A/G | 1.2 |
| | A/A | 2.2 |
| PLIN Z | G/G | 1.1 |
| rs8179043 | A/G | 1.2 |
| | A/A | 2.2 |
| PLIN 6 | T/T | 1.1 |
| rs1052700 | T/A | 1.2 |
| | A/A | 2.2 |
| PLIN X | G/G | 1.1 |
| rs4578621 | G/C | 1.2 |
| | C/C | 2.2 |
| PLIN 5 | T/T | 1.1 |
| rs2304795 | C/T | 1.2 |
| | C/C | 2.2 |

Predictive Medicine

Identifying PLIN Alleles and Haplotypes

The present invention is based at least in part, on the identification of certain alleles that have been determined to be in association (to a statistically significant extent) to resistance to weight-loss. Therefore, detection of the alleles can indicate that the subject has or is predisposed to resistance to weight-loss. However, because these alleles are in linkage disequilibrium with other alleles, the detection of such other linked alleles can also indicate that the subject has or is predisposed to resistance to weight-loss. For example, the haplotype (AAG) comprises the following polymorphisms:

TABLE 2

allele A of PLIN 4 (rs894160)
allele A of PLIN Z (rs8179043)
allele G of PLIN 1 (rs2289487)

In some embodiments the haplotype GGA comprises the following polymorphisms:

TABLE 3

allele G of PLIN 4 (rs894160)
allele G of PLIN Z (rs8179043)
allele A of PLIN 1 (rs2289487)

In some embodiments, the haplotype AAGG comprises the following polymorphisms:

TABLE 4

allele A of PLIN 4 (rs894160)
allele A of PLIN Z (rs8179043)
allele G of PLIN 1 (rs2289487)
allele G of PLIN X (rs4578621)

In some embodiments, the haplotype GGAG comprises the following polymorphisms:

TABLE 5

allele G of PLIN 4 (rs894160)
allele G of PLIN Z (rs8179043)
allele A of PLIN 1 (rs2289487)
allele G of PLIN X (rs4578621)

In addition to the allelic patterns described above, as described herein, one of skill in the art can readily identify other alleles (including polymorphisms and mutations) that are in linkage disequilibrium with an allele associated with resistance to weight loss. For example, a nucleic acid sample from a first group of subjects without resistance to weight loss can be collected, as well as DNA from a second group of subjects with the disorder. The nucleic acid sample can then be compared to identify those alleles that are over-represented in the second group as compared with the first group, wherein such alleles are presumably associated with resistance to weight loss. Alternatively, alleles that are in linkage disequilibrium with an allele that is associated with resistance to weight loss can be identified, for example, by genotyping a large population and performing statistical analysis to determine which alleles appear more commonly together than expected. Preferably, the group is chosen to be comprised of genetically related subjects. Genetically related subjects include subjects from the same race, the same ethnic group, or even the same family. As the degree of genetic relatedness between a control group and a test group increases, so does the predictive value of polymorphic alleles which are ever more distantly linked to a disease-causing allele. This is because less evolutionary time has passed to allow polymorphisms which are linked along a chromosome in a founder population to redistribute through genetic cross-over events. Thus race-specific, ethnic-specific, and even family-specific diagnostic genotyping assays can be developed to allow for the detection of disease alleles which arose at ever more recent times in human evolution, e.g., after divergence of the major human races, after the separation of human populations into distinct ethnic groups, and even within the recent history of a particular family line.

Linkage disequilibrium between two polymorphic markers or between one polymorphic marker and a disease-causing mutation is a meta-stable state. Absent selective pressure or the sporadic linked reoccurrence of the underlying mutational events, the polymorphisms will eventually become disassociated by chromosomal recombination events and will thereby reach linkage equilibrium through the course of human evolution. Thus, the likelihood of finding a polymorphic allele in linkage disequilibrium with a disease or condition may increase with changes in at least two factors: decreasing physical distance between the polymorphic marker and the disease-causing mutation, and decreasing number of meiotic generations available for the dissociation of the linked pair. Consideration of the latter factor suggests that, the more closely related two subjects are, the more likely they will share a common parental chromosome or chromosomal region containing the linked polymorphisms and the less likely that this linked pair will have become unlinked through meiotic cross-over events occurring each generation. As a result, the more closely related two subjects are, the more likely it is that widely spaced polymorphisms may be co-inherited. Thus, for subjects related by common race, ethnicity or family, the reliability of ever more distantly spaced polymorphic loci can be relied upon as an indicator of inheritance of a linked disease-causing mutation.

Appropriate probes may be designed to hybridize to a specific allele of the PLIN locus, such as PLIN 1, PLIN 4 or PLIN Z or a neighboring alleles. Alternatively, these probes may incorporate other regions of the relevant genomic locus, including intergenic sequences. Indeed the PLIN gene of human chromosome 15 spans some 14994 base pairs and, assuming an average of one single nucleotide polymorphism every 1,000 base pairs, includes some 149 SNPs loci alone. Yet other polymorphisms available for use with the immediate invention are obtainable from various public sources.

The present invention is further illustrated by the following examples which should not be construed as limiting in any way. The practice of the present invention will employ, unless otherwise indicated, conventional techniques that are within the skill of the art. Such techniques are explained fully in the literature. See, for example, Molecular Cloning A Laboratory Manual, (2nd ed., Sambrook, Fritsch and Maniatis, eds., Cold Spring Harbor Laboratory Press: 1989); DNA Cloning, Volumes I and II (D. N. Glover ed., 1985); Oligonucleotide Synthesis (M. J. Gait ed., 1984); U.S. Pat. No. 4,683,195; U.S. Pat. No. 4,683,202; and Nucleic Acid Hybridization (B. D. Hames & S. J. Higgins eds., 1984).

According to some embodiments, the present invention provides for methods for selecting an appropriate therapeutic/dietary regimen or lifestyle recommendation for a subject comprising: identifying in a subject's DNA polymorphism in the PLIN gene, wherein the presence of the PLIN 4 G>A, PLIN Z G>A and/or PLIN 1 A>G polymorphisms indicates that the subject is resistant to weight-loss when administered low-calorie diet, as in the current invention. Appropriate therapeutic/dietary regimen or lifestyle recommendations include, but are not limited to, calcium supplements, exercise, hormone replacement therapy, and combinations and mixtures thereof.

According to some embodiments, the present invention provides for methods for selecting an appropriate therapeutic/dietary regimen or lifestyle recommendation for a subject comprising a haplotype pattern from the group consisting of: A (rs894160 of) PLIN 4, A (rs8179043 of PLIN Z), G (rs2289487 of PLIN 1); (ii) G (rs894160 of) PLIN 4, G (rs8179043 of PLIN Z), A (rs2289487 of PLIN 1); (iii) G (rs894160 of) PLIN 4, G (rs8179043 of PLIN Z), A (rs2289487 of PLIN 1), G (rs4578621 of PLIN X); (iv) A (rs894160 of) PLIN 4, A (rs8179043 of PLIN Z), G (rs2289487 of PLIN 1), G (rs4578621 of PLIN X); wherein the presence of any one, any two, any three, or all four haplotype patterns indicates that said subject is non-responsive to diet with low calorie restriction. A symptom of resistance to weight loss is alleviated by detecting the presence of an resistance to weight loss associated genetotype and guiding medical management of obese patients with recommendations for lifestyle changes which would include diet, exercise, therapeutics, or other medical interventions that are currently used to treat the major complications of obesity, particularly metabolic syndrome and fatty liver/non-alcoholic steatohepatitis (NASH).

Associations between polymorphisms and resistance to weight loss were further evaluated by testing combinations of polymorphisms. Some specific combinations to be analyzed include alleles at PLIN 1, PLIN 4, PLIN Z, PLIN5, PLIN 6, PLIN X and PLIN Y. The analytic strategy were used to address any combination of gene polymorphisms, both for known and novel polymorphisms. Synergy among genes was assessed by adding interaction terms to a logistic regression model.

The following examples are illustrative, but not limiting, of the methods and compositions of the present invention. Other suitable modifications and adaptations of the variety of conditions and parameters normally encountered in therapy and that are obvious to those skilled in the art are within the spirit and scope of the embodiments.

### EXAMPLES

### Example 1

### A Case Control Study of Association Between PLIN Polymorphisms and Resistance to Achieve Weight Loss in Response to Low Calorie Diet

Study Design:

The main hypothesis tested was whether the sequence variations (polymorphisms) in the PLIN gene are associated with resistance to weight loss for obese subjects on energy restricted diets, potentially playing a role in the development of obesity related complications. The clinical population from the multidisciplinary preoperative surgery program at Geisinger's Center for Nutrition and Weight Management was well suited for a retrospective study to test this hypothesis. The calorie restriction regimen and the weight and metabolic parameter measured for this clinical population parallels the design of the proof-of-concept study of Corella et al (J. Clin. Endocrinol. Metab., 90(9):5121-5126, 2005) which initially identified the PLIN gene variants as potentially related to weight loss. However, the sample size in this report was only 48 subjects. The total number of subjects recruited in this study, were much larger (824) with a relatively even distribution of subjects who were “resistant” and “non-resistant” to weight loss from the energy restriction program.

The primary goal of this study was to investigate the association of PLIN gene polymorphisms (Listed in Table 1), both individually and in combination, with obese subjects who are “resistant” to weight loss in a defined program.

The following examples are illustrative, but not limiting, of the methods and compositions of the present invention. Other suitable modifications and adaptations of the variety of conditions and parameters normally encountered in therapy and that are obvious to those skilled in the art are within the spirit and scope of the embodiments.

TABLE 6
PLIN SNPs

| | | Complementary | |
|---|---|---|---|
| SNP | Base Change | Strand | rs number |
| PLIN 6 | T > A | A > T | rs1052700 |
| PLIN Y | T > G | A > C | rs894161 |
| PLIN 4 | G > A | C > T | rs894160 |
| PLIN 1 | A > G | T > C | rs2289487 |
| PLIN X | G > C | C > G | rs4578621 |
| PLIN 5 | T > C | A > G | rs2304795 |
| PLIN Z | G > A | C > T | rs8179043 |

Study Design

The Geisinger Study was performed into two main stages. In Stage 1 (˜4 months), all the enrolled subjects were recommended a diet consisting of 1200-1500 kcal and 1500-1800 kcal, for Women and Men, respectively. Subject who lost >3% weight were classified as group A. In Stage 2 (˜4 months) all the subjects that lost <3% weight in Stage 1, were recommended a liquid diet of 1000 kcal and 1200 kcal, for Women and Men, respectively. Once on liquid diet, subjects who lost >5% of total body weight early on were classified as Group B (Early responders) and people who lost the same weight but at a later stage were put in Group C (Late responders). Subjects who did not respond to the low calorie diet in Stage 1 but who did lose weight on the very low calorie liquid diet that was high in protein and very low in fat were classified as Group BC. Subjects, who did not respond to either of the diets, were classified as Group D (Non-responders).

Body weight, lipid profile and other metabolic parameters were measured for all the enrolled subjects during their site visits.

Cases and Controls

824 subjects were evaluated at baseline. 372 subjects responded to low calorie diet with 4 months, and were classified in Group A, whereas 93 were classified in Group B (early responders of liquid diet for 120 days), and additional 92 were in Group C (late responders of liquid diet for 120 days). 185 subjects were classified as Group BC. 267 subjects did not respond, i.e., lost less than 5% of body weight after being on liquid calorie diet for 120 days, and were classified in D category (controls). The overall study design is shown in FIG. 1.

In this study, a subject was classified as weight loss “resistant” based on a failure to lose 3% of their baseline bodyweight on dietary modification counseling designed to reduce caloric intake by 500 kcal, and if unsuccessful with diet modification, failure to lose the 5% on a prescription 1000 kcal liquid diet.

Subjects who were successful in weight loss were divided into two groups: (a) Low calorie (Group A): those who lost weight on the recommended diet of 500 kcal deficit from estimated calories consumed daily and, (b) Liquid diet (Group BC): those who were initially resistant to weight loss with the above dietary plan for the first 4 months but eventually achieved weight loss when put on the very low calorie (1000-1200 kcal) liquid diet that was high in protein and very low in fat.

Sample Collection and Statistical Parameters

DNA samples obtained from the subjects were genotyped for the selected SNPs in PLIN gene (Table 1). Genetic association of PLIN SNPs with body weight loss in low calorie diet responders versus non-responders was analyzed by logistical regression analysis in group-wise comparisons adjusting the data for age, gender, antidepressant and diabetic medications, statins and diuretics. Genetic association was also analyzed for the lipid profile and metabolic parameters (Quantitative Traits) in a linear regression analysis using additive, dominant and recessive genetic models and adjusting for age, gender, metabolic score (co-morbidities), metformin, statins, antidepressant and diabetic medications. Data analysis was performed for three categories, Full data and two age stratified groups, Young Age (<47.5 year old) and Old Age (>47.5 year old).

Genotyping

Peak Migration

Each SNP-specific single base extension primer was designed at a unique length to create a peak(s) at a specific location in relation to the known size standards when run on the CEQ8800 capillary electrophoresis instrument. The peak locations may not exactly match the primer sizes due to the effects of dye mobility, primer sequence and the analysis software, but they do migrate consistently. Single base extension primers are listed in Appendix C along with their expected peak migrations.

Base Calling

The single base extension reaction adds a fluorescently labeled base to the 3′ end of the SNP-specific primer. This product is read by two lasers within the CEQ8800. The results are analyzed by the CEQ8800 software and appear as colored peaks—each color representing a different base. Presence of one single-colored peak at the specified locus indicates a homozygote while two peaks of different colors indicate a heterozygote. Within the thirty-nine samples that were genotyped in the validation are representatives of almost all homozygous and all heterozygous genotypes for all five SNPs. The one exception is a homozygous C genotype for the PPARG SNP. This was not unexpected since the frequency of the C allele in the general population is only 0.1 (as indicated by the dbSNP database for rs#1801282). However, the homozygous C genotype has been encountered in other samples outside the scope of this validation.

The CEQ8800 software features the ability for the user to specify SNP locus tags. The user indicates the migration size (in nucleotides) based on the expected migration of the SNP-specific primer. This enables the computer to identify a SNP based on its migration in relation to the standardized markers run along with the sample. The computer will also identify the base(s) within the SNP based on the dye indicator(s) it detects. For this validation, the computer was allowed to make the initial call of each SNP. The data was then independently re-analyzed by two technicians for confirmation. In all cases the computer calls and the two independent (manual) calls were in agreement.

Statistical Analyses

Summary statistics was provided as means and their standard error for normal continuous variables and medians and inter-quartile range for continuous non-normal variables. Frequencies were provided for categorical variables. For non-normal variables log-transformations were applied. For continuous variables to test the mean differences of difference responses ANOVA method was applied and for categorical variables fisher exact test was used.

Standard quality control methods were used to check for genotyping error call rate, minor allele frequency etc., within locus intendance was tested using Hardy-Weinberg equilibrium with fisher exact test. Covariates were identified for each continuous (dichotomous) phenotype using multiple linear (logistic) regression analysis with backward elimination method. For individual SNPs, multiple logistic (linear) regression analysis was applied to test the association between SNP variants and dichotomous (continuous) phenotype after adjusting for their covariates. Permutation method was applied to adjust p-value for multiple hypotheses. Linkage Disequilibrium Plot was generated using Haploview program.

Haplotypes were inferred for tightly linked loci using FAMHAP program (Becker T and Knapp, Genetic Epidemiology, 2004, V27, pp 21-32) and association between haplotypes (diplotyeps) and phenotypes was tested using linear/logistic regression analysis after adjusted for covariates. Genetic association analysis were performed using PLINK software.

TABLE 7

| Characterisristic | Group A | | Group B | | Group C | | Group D | | p-value |
|---|---|---|---|---|---|---|---|---|---|
| Number of Patients | 372 | | 93 | | 92 | | 267 | | |
| Initial mean BMI (SD) | 51.0 | (8.3) | 49.6 | (7.8) | 49.9 | (8.5) | 48.4 | (7.2) | 0.000930* |
| Mean Age, yrs | 47.5 | (11.2) | 45.6 | (9.8) | 47.5 | (6.7) | 43.8 | (10.6) | 0.000235* |
| Males (#) | 68 | (18.3%) | 25 | (27.9%) | 23 | (25%) | 54 | (20.2%) | 0.205734 |
| Females (#) | 304 | (81.7%) | 68 | (73.3%) | 69 | (75%) | 213 | (79.8%) | |
| Age > 47.5 (#) | 200 | (53.8%) | 38 | (40.9%) | 49 | (53.3%) | 94 | (35.2%) | 0.00002 |
| Age < 47.5 (#) | 172 | (46.2%) | 55 | (59.1%) | 43 | (46.7%) | 173 | (64.8%) | |

| | | | | | |
|---|---|---|---|---|---|
| Hypertension | 174 | 43 | 40 | 117 | 0.869549 |
| Diabetes | 132 | 35 | 47 | 83 | 0.007278 |
| Hypercholesterolemia | 137 | 38 | 37 | 99 | 0.847789 |
| General symptoms | 124 | 29 | 20 | 91 | 0.150327 |
| Depressive disorder | 83 | 25 | 25 | 77 | 0.285318 |
| Oesophageal disease | 95 | 21 | 22 | 73 | |
| Osteoarthritis | 95 | 19 | 18 | 50 | 0.185914 |
| Asthma | 45 | 12 | 11 | 33 | 0.996596 |
| Family history of diabetes | 17 | 8 | 1 | 28 | 0.002569 |
| Affective psychoses | 14 | 4 | 0 | 10 | 0.293739 |

Linkage Disequilibrium (LD) Plot.

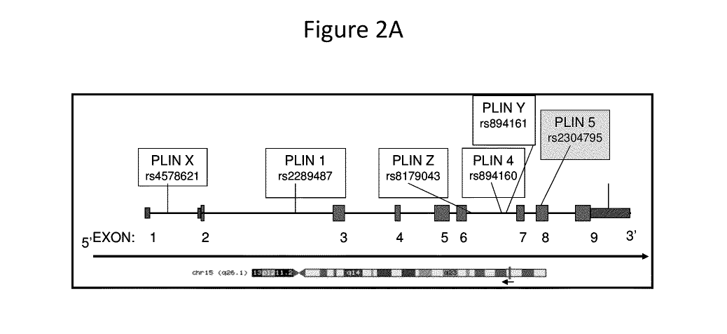

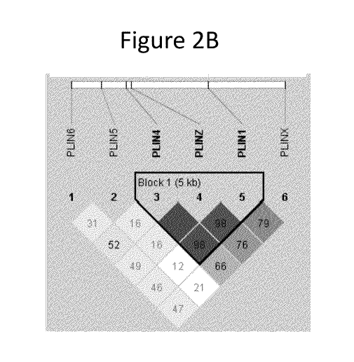

Linkage disequilibrium (LD) plot were generated in Haploview software (r² shown) for all SNPs. Strong LD between SNPs PLIN 1, PLIN 4 and PLIN Z was observed. See FIG. 2B.

The table (Table 9) below provides data showing association of PLIN 1, PLIN 4 and PLIN Z SNPs with weight loss under different diets

TABLE 8

| | | | | | | | | | | |
|---|---|---|---|---|---|---|---|---|---|---|
| | Comparison | Associated | | Freq in non | Freq in | | | | p-Value | p-Value |
| SNP | Group | genotype | Test | responders | responders | OR (Adj) | L95 (Adj) | U95 (Adj) | (Adj) | (Perm) |

A_BC (low calorie diet responders vs liquid diet responders)

| | | | | | | | | | | |
|---|---|---|---|---|---|---|---|---|---|---|
| PLIN4 | Full data | A/A | REC | 0.3135 | 0.2769 | 2.03 | 1.065 | 3.868 | 0.0313 | 0.0214 |
| PLINZ | | A/A | REC | 0.3216 | 0.2863 | 1.849 | 0.9919 | 3.445 | 0.0531 | 0.0461 |

A_D (low calorie diet responders vs resistant)

| | | | | | | | | | | |
|---|---|---|---|---|---|---|---|---|---|---|
| PLIN1 | OLD | G/\hat{ } | DOM | 0.3191 | 0.3725 | 1.6835 | 1.0037 | 2.8233 | 0.0484 | 0.0467 |

In Table 8, “non-responders’ are patients who did not achieve the target level of weight loss after the specific dietary intervention period, e.g. A vs, BC means that Group A subjects achieved the targeted magnitude of weight loss after the Stage 1 diet and Group BC are subjects who did not achieve weight loss after Stage 1 but did successfully lose the targeted magnitude of weight loss after the Stage 2 diet. Therefore, Group BC would be considered the “non-responsive group” after Stage 1.

a. Those subjects who did not lose weight on the low calorie Stage 1 diet but did successfully lose weight on the very low calorie and very low fat liquid diet in Stage 2 (Group BC; “non-responders”) were found to be enriched for PLIN 4 (rs894160; A/A; p=0.03; Odds ratio=2.0) and PLIN Z (rs8179043; A/A; p=0.05; Odds ratio=1.8) genotypes compared to those who successfully lost weight in Stage 1 (Group A; “responders”). Table 8 comparison A vs BC.

b. Those subjects who did not successfully lose weight on either Stage 1 or Stage 2 diets were considered non-responders (Group D; “non-responders”). Subjects who were likely to lose weight successfully on the low calorie Stage 1 diet (Group A; “responders”) were enriched for PLIN 1 (rs2289487); A/A; p=0.048), meaning that subjects with the PLIN 1 (rs2289487) G/* genotype will be less likely to successfully lose weight with either diet. Table 8 comparison A vs D. The position of all the tested SNPs on PLIN gene and their LD analysis is shown in the figures below (FIGS. 2 A and B).

### Example 2

### A Case Control Study of Association Between PLIN Haplotype and Resistance to Achieve Weight Loss in Response to Low Calorie Diet

Logistic regression analysis of the haplotypes on PLIN gene in the responders versus non-responders to calorie restricted diet showed an association two haplotypes in the old age group. The results are shown in the table below (Table 9).

TABLE 9

| | | | | | | | | |
|---|---|---|---|---|---|---|---|---|
| | | Comparison | | | | | | |
| Gene | No. of SNPs | Group | Haplotype | OR | L95 | U95 | P | SNPs |

A_BC: (Low calorie diet responders versus liquid diet responders)

| | | | | | | | | |
|---|---|---|---|---|---|---|---|---|
| PLIN | 3 | Old Age | AAG/GGG | 3.39 | 1.23 | 9.3 | 0.018 | 4/Z/1 |
| | 3 | | GGA/GGG | 3.02 | 1.12 | 8.12 | 0.025 | 4/Z/1 |
| | 4 | | GGAG/GGGG | 2.98 | 1.1 | 8 | 0.031 | 4/Z/1/X |
| | 4 | | AAGG/GGGG | 3.4 | 1.22 | 9.5 | 0.018 | 4/Z/1/X |

Two haplotype patterns consisting of 3 SNPs (PLIN 4, PLIN Z and PLIN 1; AAG or GGA) and 4 SNPs (PLIN 4, PLIN Z, PLIN 1 and PLIN X; GGAG or AAGG) were associated with the weight loss outcome. Those subjects who did not lose weight on the low calorie Stage 1 diet but did successfully lose weight on the very low calorie and very low fat liquid diet in Stage 2 (Group BC; “affected”) were found to be enriched for haplotype patterns, AAG or GGA in the 3 SNPs and GGAG or AAGG in the 4 SNPs category, respectively.

### CONCLUSION

PLIN genotypes were associated with resistance to weight loss on diet with moderate calorie restriction (Group A)

PLIN 4 genotype AA and PLIN Z genotype AA were associated with resistance to weight loss on diet with moderate calorie restriction (Group A) compared to liquid diet (1,000 to 1,200 kcal) (Group BC), but were responsive to weight loss on a very low calorie, low fat liquid diet.

### Example 3

### Perilipin Genotypes Interact with Diet to Influence Weight Loss in the CALERIE Study

The methods and results for the initial CALERIE study are published in the Am J Clin Nutr (2007); 85:1023-30, which is incorporated herein by reference in its entirety. Briefly, the CALERIE study was designed to observe the long-term effects of two diets varying in glycemic content over a one year period.

In a pilot study of the CALERIE trial 47 overweight men and women, aged 20-42 years and having a BMI of 25-30 kg/m², were subjected to three separate trial phases. Phase 1 was used to measure weight-maintenance energy requirements of the individuals in the study. In Phase 2, the subjects were randomized to two separate treatment arms: (1) 30% caloric restriction and (2) 10% caloric restriction. Each of these treatment arms was further sub-divided into a group receiving a high glycemic (HG) diet and a group receiving a low glycemic (LG) diet, however the total caloric value was the same in each treatment arm. In Phase 2, the food was provided to the participants of the study for 6 months. In Phase 3 of the pilot study, the participants were counseled on food selection and total caloric intake, however participants self-selected their food during the 6 months of Phase 3, while maintaining a similar caloric intake and appropriate glycemic diet for their treatment arm. The participants on the high glycemic diet received a diet comprising 60% carbohydrates, 20% protein, 20% fat, 15 g/1000 kcal fiber and having an energy density of 1 kcal/g. The high glycemic diet is therefore consistent with popular diets that are classified as “low fat.” An example of a low fat diet is the “Ornish” diet. Participants on the low glycemic diet received a diet comprising 40% carbohydrate, 30% protein, 30% fat, 15 g/1000 kcal fiber and having an energy density of 1 kcal/g. The low glycemic diet is therefore consistent with popular diets that are classified as “balanced” by some and as “low carbohydrate” by others because the percentage of calories contributed by carbohydrates is 40%. An example of the balanced diet is the “Zone” diet.

In Phase 1 of the pilot study, 47 overweight men and women were initially enrolled with one participant opting out during baseline measurements. Of the 46 remaining participants, 17 were randomly assigned to a 30% calorie restricted HG diet (of which 15 completed the study), 17 were randomly assigned to a 30% caloric restriction LG diet (of which 14 completed the study) and 12 were randomized to a 10% caloric restriction (of which 10 completed the study). For purposes of analysis of genotype interaction with macronutrients, we analyzed only the 30% caloric restriction groups: HG (Low Fat; n=15) and LG (Balanced; n=14).

Subjects in the HG diet had a mean age of 34±5 years, a mean BMI of 27.5±1.6 kg/m², a mean height of 169.1±10.7 cm, a mean weight of 79.0±12.1 kg and a mean % body fat of 34.8±7.1%. Subjects in the LG diet group had a mean age of 35±6 years, a mean BMI of 27.6±1.2 kg/m², a mean height of 169.0±10.2 cm, a mean weight of 79.1±9.2 kg and a mean % body fat of 34.9±8.2%. There were no statistically significant differences between the two groups in any of the aforementioned parameters.

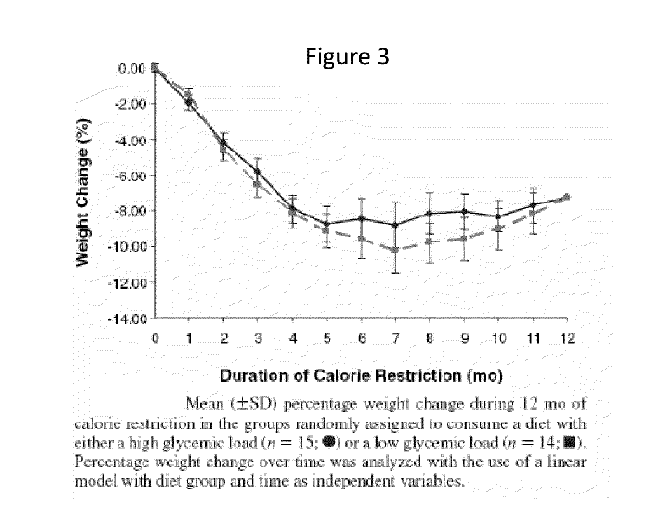

All treatment groups in the CALERIE pilot study consumed less energy during caloric restriction than at baseline (p<0.01). The changes in energy intake, body weight, body fat, and resting metabolic rate did not significantly change between the treatment groups. Subjects in the HG diet group lost an average of 8.04±4.1% weight after 6 months, while subjects in the LG diet group lost an average of 7.8±5.0% after 6 months. FIG. 3 shows the percent weight change during 12 months of calorie restriction in the groups randomly assigned to consume a diet with either a high glycemic load, or a low glycemic load. Table 10 shows calorie intake measurements for the HG and LG diets during caloric restriction, and Table 11 shows the resting metabolic rate and body composition in the two diet groups.

TABLE 10
Prescribed energy intake during calorie restriction (CR)
and energy intake expressed as a percentage of baseline
total energy expenditure (TEE) at 3, 6, and 12 mo of CR¹

| | HG diet | LG diet |
|---|---|---|
| | (n = 15) | (n = 14) |
| Baseline TEE (kcal/d) | 2825 ± 499 | 2708 ± 373 |
| Prescribed energy intake (kcal/d) | 1960 ± 364 | 1900 ± 251 |
| Measured CR at 3 mo (%)² | 21.1 ± 10.3 | 27.5 ± 13.0 |
| Measured CR at 6 mo (%)² | 15.7 ± 12.7 | 17.5 ± 15.3 |
| Measured CR at 12 mo (%)² | 17.1 ± 13.0 | 9.5 ± 14.2 |

¹All values are x̅ ± SD. HG, high glycemic load; LG, low glycemic load.
²There was a statistically significant difference over time (P < 0.01) but not between groups (P = 0.922) (mixed-model analysis of repeated measures). There was no significant diet-by-time interaction (P = 0.125).

Table 11 shows the resting metabolic rate and body composition in the two diet groups.

TABLE 11
Resting metabolic rate (RMR) and body
composition in the 2 diet groups¹

| | | Change from baseline | |
|---|---|---|---|
| | Baseline² | 6 mo | 12 mo |
| | % | | |
| Body weight (kg)³ | | | |
| HG diet (n = 15) | 78.5 ± 12.3 | −9.1 ± 4.2 | −8.0 ± 4.1 |
| LG diet (n = 14) | 78.0 ± 9.3 | −10.4 ± 4.1 | −7.8 ± 5.0 |
| Body fat (%)³ | | | |
| HG diet (n = 15) | 35.0 ± 7.1 | −17.1 ± 11.6 | −14.8 ± 8.8 |
| LG diet (n = 14) | 35.2 ± 8.7 | −23.3 ± 16.6 | −17.9 ± 12.5 |
| RMR (kcal/d)⁴ | | | |
| HG diet (n = 15) | 1582 ± 255 | −5.9 ± 5.7 | −3.3 ± 7.1 |
| LG diet (n = 14) | 1605 ± 182 | −6.6 ± 5.6 | −2.2 ± 7.8 |

¹All values are χ̅ ± SD, CR, calorie restriction; HG, high glycemic load; LG, low glycemic load.
²There were no statistically significant differences between the groups (independent-sample t tests).
^{3,4}There was a statistically significant change over time; ³P < 0.001, ⁴P < 0.01. There were no statistically significant differences between the diet groups over time (mixed-model analysis of repeated measures). There was no significant diet-by-time interaction.

A follow-up study was performed on 30 subjects to test their genotype at the perilipin locus. 29 of the subjects had completed 6 and 12 months data time-points, while one subject dropped out in the early stages of the study prohibiting the use of that subject's data in the follow-up study. Of the 30 cheek swabs received 12 were in the 30% caloric restriction HG diet group, 11 were in the 30% caloric restriction LG diet group, 3 were in the 10% caloric restriction HG diet group and 4 were in the 10% caloric restriction LG diet group. The perilipin genotypes of subjects in the CALERIE study are shown herein in Table 12. The numbers along the top of Table 12 refer to the alleles present and allele 2 is used herein to denote the minor allele of each PLIN SNP. For example, a wild-type individual has two copies of allele 1 and is denoted as 1.1, while an individual heterozygous for allele 1 and allele 2 is denoted by 1.2; it should be noted that *2 is used to denote the number of individuals who have at least one copy of allele 2 and is the total number of subjects that are heterozygous (i.e., 1.2) and homozygous for allele 2 (i.e., 2.2.). Table 13 shows the perilipin genotypes of the subjects divided into the separate treatment arms of the pilot CALERIE study.

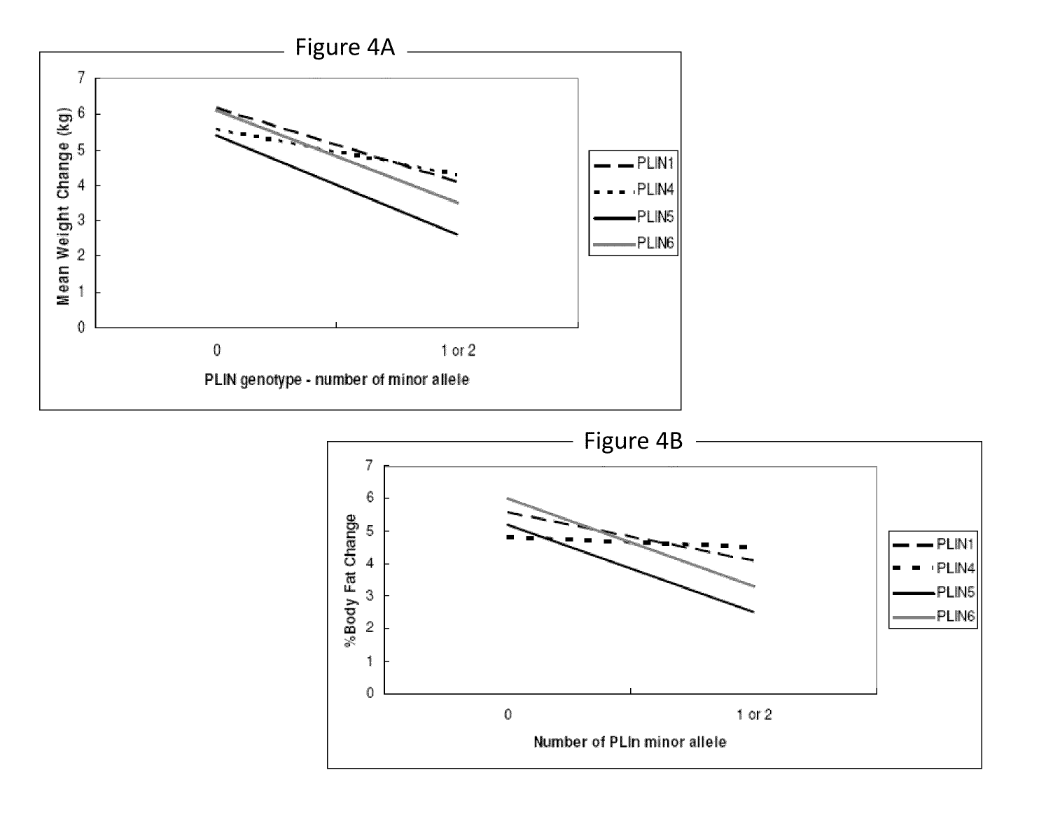

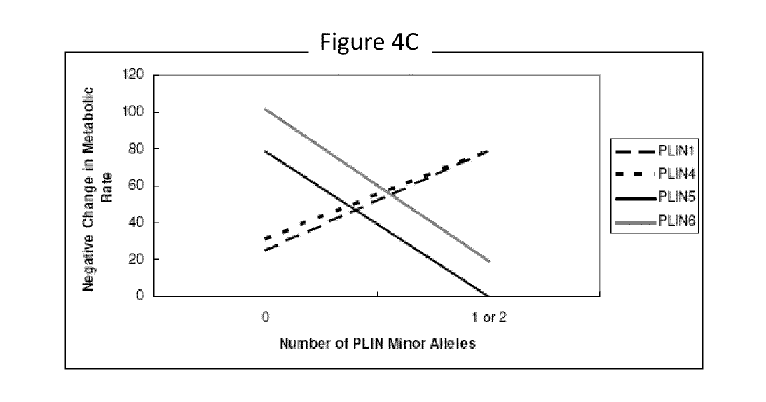

FIGS. 4A, B, C show the effect of carriage of PLIN minor alleles on the amount of weight loss (FIG. 4A), fat loss (4B) and change in metabolic rate (FIG. 4C) independent of diet typeduring the CALERIE pilot study. The steepness of the slope indicates the genotype effect on change, independent of the diet. Carriers of perilipin SNP5 (PLIN5) allele 2 (allele “C”) have the least amount of weight change and fat change at the one year time point. FIG. 4C shows the effect of calorie restriction on metabolic rate in the different PLIN genotypes. Subjects with the PLIN1 and PLIN4 minor alleles decrease in metabolic rate (i.e. higher negative change) as do the subjects who are homozygous for the wildtype of the PLIN5 and PLIN6 SNPs.

TABLE 12
Perilipin Genotype in CALERIE study

| | 1.1 | 1.2 | 2.2 | *2 |
|---|---|---|---|---|
| PLIN 1 | 12 | 11 | 6 | 17 |
| PLIN 4 | 14 | 13 | 2 | 15 |
| PLIN5 | 20 | 9 | 0 | 9 |
| PLIN6 | 13 | 14 | 2 | 16 |

TABLE 13
Perilipin Genotype in Treatment Arms

| Treatment arm | Subject # | PLIN5 *2 | PLIN5 1.1 |
|---|---|---|---|
| High carb—30% | 11 | 4 | 7 |
| Low carb—30% | 11 | 5 | 6 |
| High carb—10% | 3 | 0 | 3 |
| Low carb—10% | 4 | 0 | 4 |

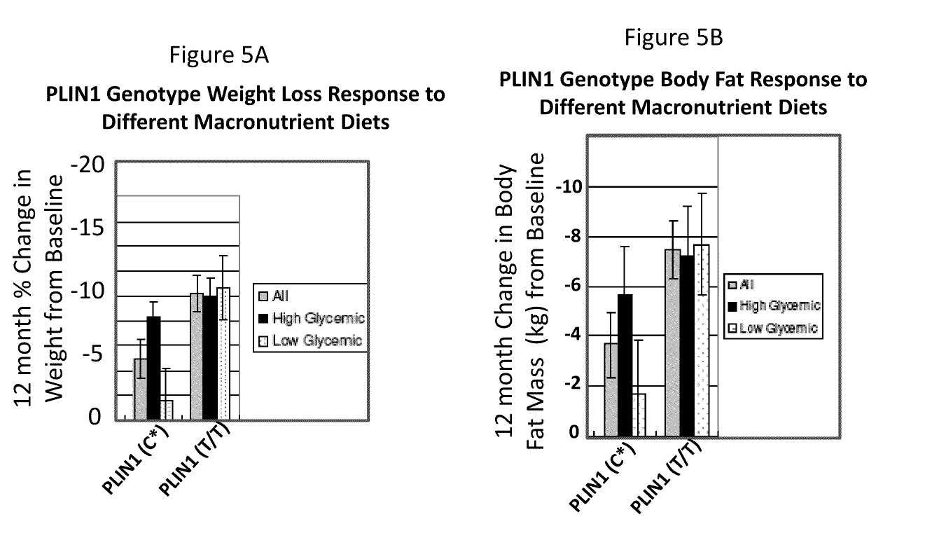

FIG. 5A shows mean weight loss over a period of one year between individuals with different PLIN1 polymorphisms assigned to either a high glycemic (Low Fat) or low glycemic diet (Balanced Diet). Individuals having a PLIN1 C* (complementary strand would read G*) genotype lost significantly (p<0.05) more weight on a high glycemic (Low Fat) diet than a low glycemic diet. Subjects who were PLIN1 T/T (complementary strand would read A/A) genotype lost comparable amounts of weight on either a HG or LG diet. As seen in FIG. 5B the change in body fat reflected the same genotype-diet interactions as noted above for percentage weight loss. Table 14 below includes the changes in weight and fat mass over time for the PLIN1 genotype interaction with diet.

TABLE 14

| | | | | | | |
|---|---|---|---|---|---|---|
| All | SE | High Glycemic | SE | Low Glycemic | SE | p-value |

PLIN-1 Association with Percent Change in Weight

| | | | | | | | |
|---|---|---|---|---|---|---|---|
| C* (3 Months) | −5.88 | 0.62 | −6.21 | 0.48 | −5.54 | 1.17 | 0.06 |
| T/T (3 Months) | −7.98 | 0.73 | −7.58 | 1.22 | −8.38 | 0.88 | |
| C* (6 Months) | −7.37 | 1.10 | −8.10 | 0.92 | −6.64 | 2.05 | 0.02 |
| T/T (6 Months) | −12.56 | 1.10 | −11.75 | 1.61 | −13.37 | 1.57 | |
| C* (9 Months) | −6.35 | 1.42 | −8.12 | 1.29 | −4.57 | 2.46 | 0.01 |
| T/T (9 Months) | −12.36 | 1.49 | −11.34 | 1.85 | −13.37 | 2.44 | |
| C* (12 Months) | −5.00 | 1.58 | −8.31 | 1.23 | −1.68 | 2.46 | 0.01 |
| T/T (12 Months) | −10.25 | 1.46 | −9.86 | 1.68 | −10.64 | 2.55 | |

PLIN-1 Assocaition with Change in Body Fat Mass

| | | | | | | | |
|---|---|---|---|---|---|---|---|
| C* (3 Months) | −4.30 | 0.47 | −4.07 | 0.45 | −4.53 | 0.85 | 0.22 |
| T/T (3 Months) | −5.17 | 0.52 | −5.08 | 0.88 | −5.26 | 0.63 | |
| C* (6 Months) | −5.67 | 0.89 | −5.52 | 0.82 | −5.82 | 1.65 | 0.05 |
| T/T (6 Months) | −9.07 | 1.01 | −8.75 | 1.65 | −9.40 | 1.31 | |
| C* (9 Months) | −4.61 | 1.05 | −4.99 | 0.86 | −4.24 | 1.08 | 0.03 |
| T/T (9 Months) | −9.01 | 1.24 | −8.87 | 1.69 | −9.15 | 1.34 | |
| C* (12 Months) | −3.65 | 1.28 | −5.63 | 1.99 | −1.66 | 2.16 | 0.02 |
| T/T (12 Months) | −7.47 | 1.16 | −7.24 | 1.96 | −7.69 | 2.03 | |

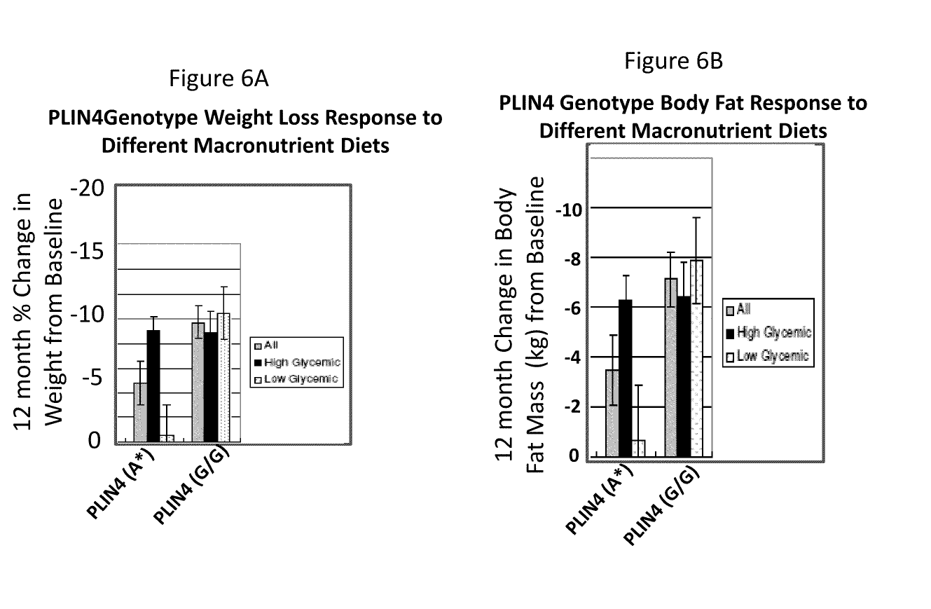

FIG. 6A shows mean weight loss over a period of one year between individuals with different PLIN4 polymorphisms assigned to either a high glycemic (Low Fat) or low glycemic diet (Balanced Diet). Individuals having a PLIN4 A* genotype lost significantly (p<0.05) more weight on a high glycemic (Low Fat) diet than a low glycemic diet. Subjects who were PLIN4 G/G genotype lost comparable amounts of weight on either a HG or LG diet. As seen in FIG. 6B the change in body fat reflected the same genotype-diet interactions as noted above for percentage weight loss. Table 15 below includes the changes in weight and fat mass over time for the PLIN4 genotype interaction with diet.

TABLE 15

| | | | | | | |
|---|---|---|---|---|---|---|
| All | SE | High Glycemic | SE | Low Glycemic | SE | p-value |

PLIN-4 Association with Percent Change in Weight

| | | | | | | | |
|---|---|---|---|---|---|---|---|
| A* (3 Months) | −5.71 | 0.63 | −6.48 | 0.46 | −4.94 | 1.15 | 0.05 |
| G/G (3 Months) | −7.85 | 0.69 | −7.12 | 1.13 | −8.58 | 0.77 | |
| A* (6 Months) | −7.11 | 1.16 | −8.57 | 0.91 | −5.65 | 2.08 | 0.02 |
| G/G (6 Month) | −12.08 | 1.09 | −10.76 | 1.68 | −13.40 | 1.33 | |
| A* (9 Months) | −6.15 | 1.52 | −8.97 | 1.12 | −3.34 | 2.46 | 0.01 |
| G/G (9 Months) | −11.69 | 1.47 | −10.03 | 2.04 | −13.35 | 2.06 | |
| A* (12 Months) | −4.80 | 1.78 | −9.03 | 1.15 | −0.56 | 2.53 | 0.02 |
| G/G (12 Months) | −9.70 | 1.34 | −8.92 | 1.70 | −10.49 | 2.16 | |

PLIN-4 Association with Change in Body Fat Mass

| | | | | | | | |
|---|---|---|---|---|---|---|---|
| A* (3 Months) | −4.11 | 0.40 | −4.30 | 0.44 | −3.93 | 0.69 | 0.14 |
| G/G (3 Months) | −5.23 | 0.55 | −4.71 | 0.84 | −5.75 | 0.73 | |
| A* (6 Months) | −5.36 | 0.88 | −5.82 | 0.89 | −4.90 | 1.58 | 0.05 |
| G/G (6 Month) | −8.90 | 0.98 | −7.99 | 1.59 | −9.81 | 1.18 | |
| A* (9 Months) | −4.35 | 1.11 | −5.38 | 0.89 | −3.33 | 2.04 | 0.06 |
| G/G (9 Months) | −8.64 | 1.17 | −7.93 | 1.72 | −9.36 | 1.67 | |
| A* (12 Months) | −3.46 | 1.40 | −6.27 | 1.00 | −0.64 | 2.20 | 0.04 |
| G/G (12 Months) | −7.11 | 1.09 | −6.37 | 1.43 | −7.85 | 1.72 | |

### Example 4

### Perilipin Genotypes Interact with Diet to Influence Body Weight Parameters in the Boston-Puerto Rican Center Study

Described herein in this example is a prospective 2 year cohort study wherein 1200 individuals of Puerto Rican descent (ages 45-75) were studied to determine the effect of stressors on allostatic load and disease. As used herein the term “allostatic load” refers to the cumulative stress on the body.

Table 16 shows the baseline characteristics for 945 subjects and Table 17 shows the incidence of disease states in these individuals. Tables 18 and 19 show preliminary data on various parameters that contribute to allostatic load of an individual. Table 20 shows the preliminary dietary data for participants in the Boston-Puerto Rican Study.

TABLE 16
Baseline Characteristics for 945 subjects in Mean (SD) or Percent

| | |
|---|---|
| Male (N = 285) | Female (N = 725) |

| Age (years) | 57.4 | (7.7) | 57.9 | (7.1) |
|---|---|---|---|---|
| Income ($) | 20,028 | | 15,538 | |
| Acculturation Score | 32.7 | (21.9) | 25.7 | (21.8) |
| Physical Activity Score | 32.7 | (6.3) | 31.2 | (4.3) |
| BMI | 30.2 | (9.6) | 33.0 | (7.6) |

| | | |
|---|---|---|
| Alcohol (% current) | 55.2 | 56.4 |
| Smoking (% current) | 46.8 | 41.6 |

TABLE 17
Preliminary Health Outcomes

| | Male (%) | Female (%) |
|---|---|---|
| Diabetes | 38.2 | 41.0 |
| High Blood Pressure | 60.4 | 60.9 |
| Heart Disease | 11.6 | 14.1 |
| Depression | 38.2 | 59.5 |
| Metabolic Syndrome* | 42.1 | 52.7 |

TABLE 18
Preliminary Data on Allostatic Load
(Percent in upper or lower quartile)

| Variable | Male | Female |
|---|---|---|
| Allostatic Load Score | 4.4 | 4.6 |
| Systolic BP ≧ 148 | 27.2 | 28.9 |
| Diastolic BP ≧ 83 | 32.6 | 49.3 |
| WHR ≧ 0.94 | 50.7 | 76.3 |
| Total Cholesterol/HDL ≧ 5.9 | 17.4 | 25.2 |
| HDL ≦ 37 | 42.7 | 53.0 |

TABLE 19
Preliminary Data on Allostatic Load
(percent in upper or lower quartile)

| Variable | Male | Female |
|---|---|---|
| GlyHgb ≧ 7.1 | 70.6 | 76.0 |
| Cortisol ≧ 25.7 | 70.6 | 52.9 |
| Norepinephrine ≧ 48 | 48.3 | 29.1 |
| Epinephrine ≧ 5 | 55.4 | 40.6 |
| DHEAS ≦ 350 | 30.2 | 26.7 |
| CRP > 4.6 | 25.8 | 29.2 |

TABLE 20
Preliminary Data on Diet

| | Male (N = 285) | Female (N = 725) |
|---|---|---|
| Energy | 2704.6 ± 86.1 | 2142.3 ± 44.1 |
| Protein | 115.6 ± 3.7 | 91.9 ± 2.0 |
| Carbohydrate | 325.3 ± 10.5 | 275.9 ± 6.0 |
| Total Fat | 101.5 ± 3.6 | 77.3 ± 1.7 |
| Saturated Fat | 31.6 ± 1.3 | 23.7 ± 0.6 |
| MUFA | 36.1 ± 1.4 | 27.0 ± 0.6 |
| PUFA | 22.9 ± 0.8 | 17.5 ± 0.4 |
| Dietary Fiber | 18.5 ± 0.6 | 16.3 ± 0.4 |
| Cholesterol | 429.0 ± 16.1 | 302.7 ± 7.5 |
| Alcohol | 8.3 ± 1.6 | 1.5 ± 0.2 |

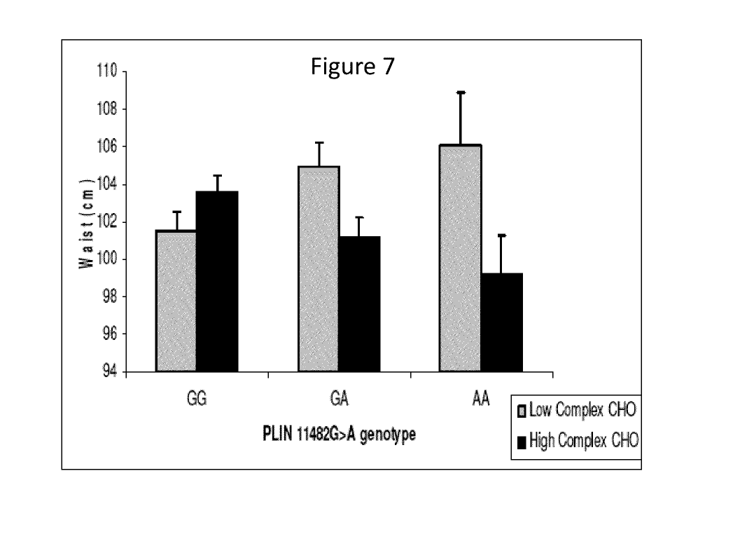

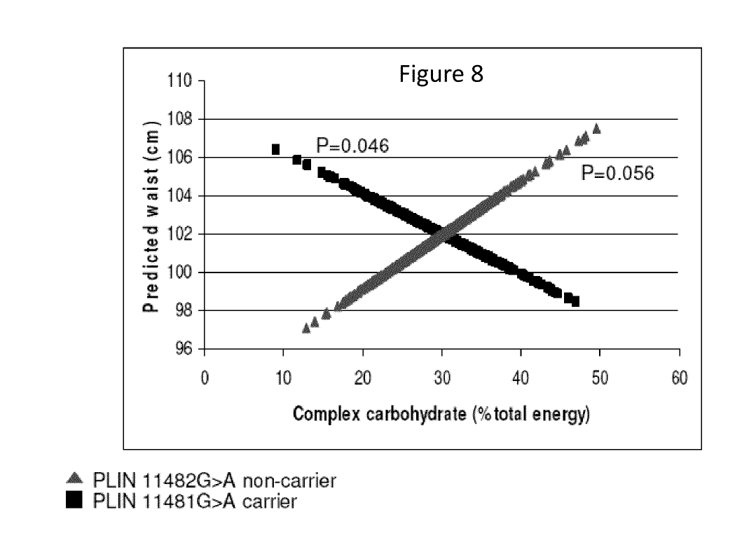

FIG. 7 shows the effect of PLIN4 polymorphism and either a high complex carbohydrate (which also results in a low fat diet, in terms of percentage of calories contributed by fats, carbohydrates, or protein) or a low complex carbohydrate diet on waist circumference of individuals. In individuals having at least one “A” allele the waist circumference is decreased only in individuals on a high carbohydrate diet—i.e. a low fat diet. FIG. 8 shows there is an inverse relationship between predicted waist size with an increasing amount of complex carbohydrate (i.e. a decreasing amount of fat) in the diet of an individual that carries the “A” allele of PLIN4. Conversely, individuals without an “A” allele (i.e., wild-type) show a linear relationship between predicted waist circumference and amount of complex carbohydrates in the diet.

What is claimed is:

1. A method for selecting an appropriate therapeutic/dietary regimen or lifestyle recommendation for a subject comprising genotyping said subject at one or more alleles selected from the group consisting of: PLIN 4, PLIN Z, PLIN 1, and PLIN 6, wherein the presence of one or more alleles is predictive of said subject's predisposition to weight loss in response to low calorie diet, or liquid diet, or both.

2. The method of claim 1, wherein selecting an appropriate therapeutic/dietary regimen or lifestyle recommendation for said subject comprises genotyping said subject at the SNP rs894160 of PLIN 4, wherein the presence of allele A indicates said subject is resistant, and presence of allele G indicates said subject is predisposed to respond to weight loss in response to a low calorie diet.

3. The method of claim 1, wherein selecting an appropriate therapeutic/dietary regimen or lifestyle recommendation for said subject comprises genotyping said subject at the SNP rs8179043 of PLIN Z, wherein the presence of allele A indicates said subject is resistant, and presence of allele G indicates said subject is predisposed to respond to weight loss in response to a low calorie diet.

4. The method of claim 1, wherein selecting an appropriate therapeutic/dietary regimen or lifestyle recommendation for said subject comprises genotyping said subject at the SNP rs2289487 of PLIN 1, wherein the presence of allele G indicates said subject is resistant, and presence of allele A indicates said subject is predisposed to respond to weight loss in response to a low calorie diet, or a liquid diet, or both.

5. A method of determining if a subject is resistant to weight loss, comprising genotyping said subject at one or more alleles selected from the group consisting of: PLIN 4, PLIN Z, PLIN 1, and PLIN6, wherein the presence of one or more alleles is predictive of said subject's predisposition to weight loss in response to low calorie diet, or liquid diet, or both.

6. The method of claim 5, wherein determining if said subject is resistant to weight loss comprises genotyping said subject at the SNP rs894160 of PLIN 4, wherein the presence of allele A indicates said subject is resistant, and presence of allele G indicates said subject is predisposed to respond to weight loss in response to a low calorie diet.

7. The method of claim 5, wherein determining if said subject is resistant to weight loss comprises genotyping said subject at the SNP rs8179043 of PLIN Z, wherein the presence of allele A indicates said subject is resistant, and presence of allele G indicates said subject is predisposed to respond to weight loss in response to a low calorie diet.

8. The method of claim 5, wherein determining if said subject is resistant to weight loss comprises genotyping said subject at the SNP rs2289487 of PLIN 1, wherein the presence of allele G indicates said subject is resistant, and presence of allele A indicates said subject is predisposed to respond to weight loss in response to a low calorie diet, or a liquid diet, or both.

9. A method for selecting an appropriate therapeutic/dietary regimen or lifestyle recommendation for a subject, comprising genotyping said subject for composite genotype at one or more alleles selected from the group consisting of: PLIN 4, PLIN Z, PLIN 1, and PLIN X, wherein the presence of one or more said composite genotypes including said alleles is predictive of said subject's predisposition to weight loss in response to low calorie diet, or liquid diet, or both.

10. The method of claim 9, wherein selecting an appropriate therapeutic/dietary regimen or lifestyle recommendation for said subject comprises the steps of:
a) genotyping said subject at:
(i) SNP rs894160 of PLIN 4;
(ii) SNP rs8179043 of PLIN Z; and
(iii) SNP rs2289487 of PLIN 1;
b) determining whether said subject has a composite genotype comprising the allelic pattern or haplotype of: allele A at SNP rs894160 of PLIN 4, allele A at SNP rs8179043 of PLIN Z, and allele G at SNP rs2289487 of PLIN 1; wherein the presence of the haplotype indicates said subject is resistant to weight loss in response to a low calorie diet.

11. The method of claim 9, wherein selecting an appropriate therapeutic/dietary regimen or lifestyle recommendation for said subject comprises the steps of:
a) genotyping said subject at:
(i) SNP rs894160 of PLIN 4;
(ii) SNP rs8179043 of PLIN Z; and
(iii) SNP rs2289487 of PLIN 1;
b) determining whether said subject has a composite genotype comprising the allelic pattern or haplotype of: allele G at SNP rs894160 of PLIN 4, allele G at SNP rs8179043 of PLIN Z, and allele A at SNP rs2289487 of PLIN 1; wherein the presence of the haplotype indicates said subject is resistant to weight loss in response to a liquid diet.

12. The method of claim 9, wherein selecting an appropriate therapeutic/dietary regimen or lifestyle recommendation for said subject comprises the steps of:
a) genotyping said subject at:
(i) SNP rs894160 of PLIN 4;
(ii) SNP rs8179043 of PLIN Z;
(iii) SNP rs2289487 of PLIN 1; and
(iv) SNP rs4578621 of PLIN X;
b) determining whether said subject has a composite genotype comprising the allelic pattern or haplotype of: allele G at SNP rs894160 of PLIN 4, allele G at SNP rs8179043 of PLIN Z, allele A at SNP rs2289487 of PLIN 1, and allele G at SNP rs4578621 of PLIN X; wherein the presence of the haplotype indicates said subject is resistant to weight loss in response to a low calorie diet.

13. The method of claim 9, wherein selecting an appropriate therapeutic/dietary regimen or lifestyle recommendation for said subject comprises the steps of:
a) genotyping said subject at:
(i) SNP rs894160 of PLIN 4;
(ii) SNP rs8179043 of PLIN Z;
(iii) SNP rs2289487 of PLIN 1; and
(iv) SNP rs4578621 of PLIN X;
b) determining whether said subject has a composite genotype comprising the allelic pattern or haplotype of: allele A at SNP rs894160 of PLIN 4, allele A at SNP rs8179043 of PLIN Z, allele G at SNP rs2289487 of PLIN 1, and allele G at SNP rs4578621 of PLIN X; wherein the presence of the haplotype indicates said subject is resistant to weight loss in response to a low calorie diet.

14. A method for selecting patients for clinical trials comprising genotyping said subject at one or more alleles selected from the group consisting of: SNP rs894160 of PLIN 4, SNP rs8179043 of PLIN Z, SNP rs2289487 of PLIN 1, SNP rs4578621 of PLIN X, and SNP rs1052700 of PLIN 6; wherein the presence of one or more alleles is predictive of said subject's predisposition to weight loss in response to low calorie diet, or liquid diet, or both.

15. A method for selecting patients for clinical trials comprising genotyping said subject for composite genotype at one or more alleles selected from the group consisting of: SNP rs894160 of PLIN 4, SNP rs8179043 of PLIN Z, SNP rs2289487 of PLIN 1, SNP rs4578621 of PLIN X, and SNP rs1052700 of PLIN 6; wherein the presence of one or more said composite genotypes including said alleles is predictive of said subject's predisposition to weight loss in response to low calorie diet, or liquid diet, or both.

16. A kit for determining a subject's response to low calorie or liquid diet toward achieving weight loss comprising reagents and instructions for genotyping said subject at one or more alleles selected from the group consisting of: SNP rs894160 of PLIN 4, SNP rs8179043 of PLIN Z, SNP rs2289487 of PLIN 1, SNP rs4578621 of PLIN X, and SNP rs1052700 of PLIN 6; wherein the presence of one or more alleles is predictive of said subject's predisposition to weight loss in response to low calorie diet, or liquid diet, or both.

17. The kit according to claim 16, wherein determining said subject's response to low calorie or liquid diet toward achieving weight loss comprises reagents and instructions for detecting in said subject allele A at SNP rs894160 of PLIN 4, wherein the reagents comprises primers, buffers, salts for detecting said allele.

18. The kit according to claim 16, wherein determining said subject's response to low calorie or liquid diet toward achieving weight loss comprises reagents and instructions for detecting in said subject allele A at SNP rs8179043 of PLIN Z, wherein the reagents comprises primers, buffers, salts for detecting said allele.

19. The kit according to claim 16, wherein determining said subject's response to low calorie or liquid diet toward achieving weight loss comprises reagents and instructions for detecting in said subject allele G at SNP rs2289487 of PLIN 1, wherein the reagents comprises primers, buffers, salts for detecting said allele.

20. A kit for determining a subject's response to low calorie or liquid diet toward achieving weight loss comprising reagents and instructions for genotyping said subject for composite genotype at one at one or more alleles selected from the group consisting of: SNP rs894160 of PLIN 4, SNP rs8179043 of PLIN Z, SNP rs2289487 of PLIN 1, SNP rs4578621 of PLIN X, and SNP rs1052700 of PLIN 6; wherein the presence of one or more alleles is predictive of said subject's predisposition to weight loss in response to low calorie diet, or liquid diet, or both.

21. The kit according to claim 20, wherein determining said subject's composite genotype, comprises reagents and instructions for:
a) genotyping said subject at:
(i) SNP rs894160 of PLIN 4;
(ii) SNP rs8179043 of PLIN Z; and
(iii) SNP rs2289487 of PLIN 1;
b) determining whether said subject has a composite genotype comprising the allelic pattern or haplotype of: allele A at SNP rs894160 of PLIN 4, allele A at SNP rs8179043 of PLIN Z, and allele G at SNP rs2289487 of PLIN 1; wherein the presence of the haplotype indicates said subject is resistant to weight loss in response to a low calorie or liquid diet.

22. The kit according to claim 20, wherein determining said subject's composite genotype, comprises reagents and instructions for:
a) genotyping said subject at:
(i) SNP rs894160 of PLIN 4;
(ii) SNP rs8179043 of PLIN Z; and
(iii) SNP rs2289487 of PLIN 1;
b) determining whether said subject has a composite genotype comprising the allelic pattern or haplotype of: allele G at SNP rs894160 of PLIN 4, allele G at SNP rs8179043 of PLIN Z, and allele A at SNP rs2289487 of PLIN 1; wherein the presence of the haplotype indicates said subject is resistant to weight loss in response to a low calorie or liquid diet.

23. The kit according to claim 20, wherein determining said subject's composite genotype, comprises reagents and instructions for:
a) genotyping said subject at:
(i) SNP rs894160 of PLIN 4;
(ii) SNP rs8179043 of PLIN Z;
(iii) SNP rs2289487 of PLIN 1; and
(iv) SNP rs4578621 of PLIN X;
b) determining whether said subject has a composite genotype comprising the allelic pattern or haplotype of: allele G at SNP rs894160 of PLIN 4, allele G at SNP rs8179043 of PLIN Z, allele A at SNP rs2289487 of PLIN 1 and allele G at SNP rs4578621 of PLIN X; wherein the presence of the haplotype indicates said subject is resistant to weight loss in response to a low calorie or liquid diet.

24. The kit according to claim 20, wherein determining said subject's composite genotype, comprises reagents and instructions for:
a) genotyping said subject at:
(i) SNP rs894160 of PLIN 4;
(ii) SNP rs8179043 of PLIN Z;
(iii) SNP rs2289487 of PLIN 1; and
(iv) SNP rs4578621 of PLIN X;
b) determining whether said subject has a composite genotype comprising the allelic pattern or haplotype of: allele A at SNP rs894160 of PLIN 4, allele A at SNP rs8179043 of PLIN Z, allele G at SNP rs2289487 of PLIN 1 and allele G at SNP rs4578621 of PLIN X; wherein the presence of the haplotype indicates said subject is resistant to weight loss in response to a low calorie or liquid diet.

25. The kit according to claim 20, wherein the reagents comprises primers, buffers, salts for detecting said composite genotype.
